(19) United States
(12) Patent Application Publication
(10) Pub. No.: US 2025/0134874 A1
(43) Pub. Date: May 1, 2025

(54) PHARMACEUTICAL FORMULATIONS COMPRISING 3-({[(4R)-7-{METHYL[4-(PROPAN-2-YL)PHENYL]AMINO}-3,4-DIHYDRO-2H-1-BENZOPYRAN-4-YL]METHYL}AMINO)PYRIDINE-4-CARBOXYLIC ACID

(71) Applicant: TACHYON THERAPEUTICS, INC., Houston, TX (US)

(72) Inventors: Gowri SUKUMAR, Houston, TX (US); Frank PERABO, Houston, TX (US); Andrew Xian CHEN, Houston, TX (US); Jianmin XU, Houston, TX (US); Ling-chieh CHEN, Houston, TX (US)

(21) Appl. No.: 18/722,083

(22) PCT Filed: Dec. 21, 2022
(86) PCT No.: PCT/US2022/053726

§ 371 Date: Jun. 20, 2024

Related U.S. Application Data
(60) Provisional application No. 63/293,488, filed on Dec. 23, 2021.

(51) Int. Cl. A61K 31/4433; A61K 9/16; A61K 9/19

(52) U.S. Cl. CPC A61K 31/4433; A61K 9/1635; A61K 9/1641; A61K 9/1652; A61K 9/19

(57) ABSTRACT

Provided herein are pharmaceutical formulations of the KDM4 inhibitor 3-({[(4r)-7-{methyl[4-(propan-2-yl)phenyl]amino}-3,4-dihydro-2h-1-benzopyran-4-yl]methyl}amino)pyridine-4-carboxylic acid.

### CROSS-REFERENCE TO RELATED APPLICATION

This application claims benefit of U.S. Patent Application No. 63/293,488, filed on Dec. 23, 2021, which is hereby incorporated by reference in its entirety.

### BACKGROUND OF THE INVENTION

A need exists in the art for an effective treatment of cancer and neoplastic disease. Provided herein are improved pharmaceutical formulations of the KDM4 inhibitor 3-({[(4R)-7-{methyl[4-(propan-2-yl)phenyl]amino}-3,4-dihydro-2H-1-benzopyran-4-yl]methyl}amino)pyridine-4-carboxylic acid.

### SUMMARY OF THE INVENTION

One embodiment provides a pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to milling.

One embodiment provides a pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to lyophilization, or spray drying, or a combination thereof.

### BRIEF DESCRIPTION OF THE DRAWINGS

The features of the invention are set forth with particularity in the appended claims. A better understanding of the features of the present invention will be obtained by reference to the following detailed description that sets forth illustrative embodiments, in which the principles of the invention are utilized, and the accompanying drawings of which:

FIG. 1 shows in vitro dissolution testing of the nano milled material prior to supplementing with PEG400;

FIG. 2 shows in vitro dissolution testing of the nano milled material post supplementing with PEG400;

FIG. 3 shows in vitro dissolution testing of lyophilized formulation F13A;

FIG. 4 shows in vitro dissolution testing of hot melt formulations;

FIG. 5 shows in vitro dissolution testing of additional formulations;

FIG. 6 shows in vitro dissolution testing of additional formulations; and

FIG. 7 shows in vitro dissolution testing of additional formulations.

### DETAILED DESCRIPTION OF THE INVENTION

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

### Histone Lysine Demethylase

Cancer is the second leading cause of death in the United States. It presents complex challenges for the development of new therapies. Cancer is characterized by the abnormal growth of malignant cells that have undergone a series of genetic changes that lead to growth of tumor mass and metastatic properties. Not only genetic changes, but also aberrant epigenetic regulation adds to the complexity of cancer.

Epigenetic regulation leads to transitions between transcriptionally silent heterochromatin and transcriptionally active euchromatin by the actions of enzymes that add or remove chemical marks from histones e.g., histone acetyltransferase, deacetylases, methyltransferase and histone demethylases and subsequently cause chromatin remodeling (Dimitrova et al., 2015; Rotili & Mai, 2011). Epigenetic modifications regulate essential physiological functions including nuclear functions such as programming development, activation or repression of transcription, timing and control of the cell cycle, and initiating DNA replication and repair. Epigenetic deregulation appears to be essential for cancer, given gene alteration or overexpression in epigenetic enzymes is commonly seen in cancer. Overexpression of histone demethylases has been associated with many cancer types (Black et al., 2010; Dimitrova et al., 2015; Gregory & Cheung, 2014; Rotili & Mai, 2011) and are being investigated as potential therapeutic targets.

The histone lysine demethylase known as KDM4 is an epigenetic regulator and key oncogenic driver across multiple tumor types. KDM4, in particular, removes methyl group from di- or tri-methyl Histone H3 Lysine 9 (H3K9me2/3) (Cloos, 2006; Klose 2006; Fodor, 2006), di- or tri-methyl Histone H3 Lysine 36 (H3K36me2/3) (Klose 2006; Young, 2013; Cascante 2014), and the linker di- or tri-methyl Histone H1.4 Lysine 26 (H1.4K26me2/3) (Trojer 2009). Overexpression of KDM4 can lead to downregulation of their substrates, e.g., H3K9me3 and aberrant gene activation triggering dysregulation of numerous pathways that can lead to malignant transformation, therefore, described in numerous tumor types including breast, colorectal, brain, renal, pancreatic, gastric, lung, testicular, prostate, bladder, melanoma, squamous cell, and lymphoma (Berry et al., 2012; Cloos et al., 2006; Ding et al., 2013; Kogure et al., 2013; Liu et al., 2009; Shi et al., 2011; Shin & Janknecht, 2007; Wissmann et al., 2007; Yamamoto et al., 2013; Yang et al., 2000; Young & Hendzel, 2013).

Six different isoforms (A-F) of KDM4 have been identified and KDM4A-C are structurally similar. Several studies substantiate the critical role of KDM4 isoforms in cancer progression and suggest that KDM4 isoform-selective inhibitors may not be effective since other isoforms may compensate for loss of function. To overcome these challenges, novel potent inhibitors simultaneously targeting multiple isoforms of KDM4 are needed.

### KDM4 Inhibitor

The heterocyclic KDM4 inhibitor described herein as Compound 1 refers to 3-({[(4R)-7-{methyl[4-(propan-2-yl)phenyl]amino}-3,4-dihydro-2H-1-benzopyran-4-yl]methyl}amino)pyridine-4-carboxylic acid. Compound 1 has the structure shown below and is also known as QC8222 or TACH101.

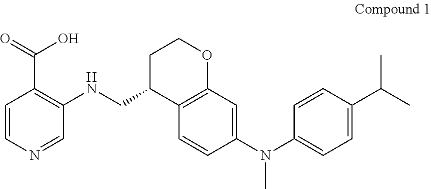 [SMILES: CC(C)c1ccc(N(C)c2ccc3c(c2)OCC[C@H]3CNc2cnccc2C(=O)O)cc1]

Compound 1 has been previously disclosed in PCT patent publication WO2015/200709 and related patent applications and granted patents, such as U.S. Pat. No. 9,242,968, which are incorporated by reference in their entirety. Compound 1 is a pan inhibitor of KDM4 that simultaneously targets multiple isoforms of KDM4. Throughout this disclosure when reference is made to a heterocyclic KDM4 inhibitor, or pharmaceutically acceptable salts or solvates thereof, the reference is to Compound 1.

There is a need in the art to provide stable, bioavailable formulations of Compound 1, wherein the formulations allow administration via oral route and are stable over prolonged period of storage.

The lysine salt of Compound 1 was selected for further study. The lysine salt of Compound 1, however, was found to have low permeability and low solubility (BCS Class IV). A BCS Class IV compound typically exhibits the lowest oral bioavailability, lowest solubility, and lowest intestinal permeability amongst all pharmaceutical classes of drugs. This class of drug product need more compatible and efficient delivery systems. Thus, it is generally recognized that BCS Class IV compounds are challenging to develop a formulation which is non-toxic and exhibits good bioavailability.

### Pharmaceutical Compositions

One embodiment provides a pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to milling. Another embodiment provides the pharmaceutical composition, wherein the milling is performed with a ball mill. Another embodiment provides the pharmaceutical composition, wherein the milling is performed with a roller mill or a high energy mill.

One embodiment provides the pharmaceutical composition wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt exhibits a particle size less than 1000 nanometers. Another embodiment provides the pharmaceutical composition wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt exhibits a particle size from about 50 nanometers to about 1000 nanometers. Another embodiment provides the pharmaceutical composition wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt exhibits a particle size from about 50 nanometers to about 100 nanometers, from about 100 nanometers to about 200 nanometers, from about 200 nanometers to about 300 nanometers, from about 300 nanometers to about 400 nanometers, from about 400 nanometers to about 500 nanometers, from about 500 nanometers to about 600 nanometers, from about 700 nanometers to about 800 nanometers, from about 800 nanometers to about 900 nanometers, or from about 900 nanometers to about 1000 nanometers. Another embodiment provides the pharmaceutical composition wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt exhibits a particle size from about 150 nanometers to about 300 nanometers.

Another embodiment provides the pharmaceutical composition wherein the particle size does not increase upon storage. Another embodiment provides the pharmaceutical composition wherein the particle size does not increase more than 5% upon storage. Another embodiment provides the pharmaceutical composition wherein the particle size does not increase more than 10% upon storage. Another embodiment provides the pharmaceutical composition wherein the particle size does not increase more than 15% upon storage.

Another embodiment provides the pharmaceutical composition wherein the at least one pharmaceutically acceptable excipient is a solubilizing agent. Another embodiment provides the pharmaceutical composition wherein the solubilizing agent is a polyethylene glycol (PEG). Another embodiment provides the pharmaceutical composition wherein the PEG is selected from PEG 200, PEG 300, PEG 400, PEG 500, or PEG 600. Another embodiment provides the pharmaceutical composition wherein the composition further comprises a stabilizer. Another embodiment provides the pharmaceutical composition wherein the stabilizer is selected from the group consisting of hydroxy propyl methyl cellulose (HPMC), hydroxypropyl cellulose (HPC), polyvinylpyrrolidone (PVP) or poloxamer.

One embodiment provides a pharmaceutical composition, comprising 3-({[(4R)-7-{methyl[4-(propan-2-yl)phenyl]amino}-3,4-dihydro-2H-1-benzopyran-4-yl]methyl}amino)pyridine-4-carboxylic acid lysine salt and at least one pharmaceutically acceptable excipient, wherein the composition comprises:
(a) 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt from about 0.1% (w/w) to about 0.2% (w/w);
(b) hydroxypropyl cellulose from about 0.91% (w/w) to about 1.5% (w/w);
(c) PEG 400 from about 5.0% (w/w) to about 10.0% (w/w); and
(d) water from about 89.3% (w/w) to about 94.4% (w/w).

Another embodiment provides the pharmaceutical composition, wherein the composition comprises:
(a) 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt 0.17% (w/w);
(b) hydroxypropyl cellulose 0.91% (w/w);
(c) PEG 400 8.32% (w/w); and
(d) water 90.49% (w/w).

Another embodiment provides the pharmaceutical composition, wherein the composition is a tablet dosage form or a capsule dosage form. Another embodiment provides the pharmaceutical composition, wherein the composition exhibits long term stability.

One embodiment provides a pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to lyophilization, or spray drying, or a combination thereof.

Another embodiment provides the pharmaceutical composition, wherein the at least one pharmaceutically acceptable excipient is a solubilizing agent. Another embodiment provides the pharmaceutical composition, wherein the solubilizing agent is a polyethylene glycol (PEG) selected from PEG 200, PEG 300, PEG 400, PEG 500, or PEG 600. Another embodiment provides the pharmaceutical composition, wherein the PEG is selected from PEG 1000, PEG 1500, or PEG 2000.

Another embodiment provides the pharmaceutical composition, wherein the at least one pharmaceutically acceptable excipient is a stabilizer. Another embodiment provides the pharmaceutical composition, wherein the stabilizer is selected from copovidone, or kollidon VA64.

Another embodiment provides the pharmaceutical composition, wherein the at least one pharmaceutically acceptable excipient is a disintegrant. Another embodiment provides the pharmaceutical composition, wherein the disintegrant is selected from crospovidone, or kollidon CL.

One embodiment provides a pharmaceutical composition, comprising 3-({[(4R)-7-{methyl[4-(propan-2-yl)phenyl]amino}-3,4-dihydro-2H-1-benzopyran-4-yl]methyl}amino)pyridine-4-carboxylic acid lysine salt and at least one pharmaceutically acceptable excipient, wherein the composition comprises:
(a) 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt from about 10% (w/w) to about 20% (w/w);
(b) Kollidon VA 64 from about 70% (w/w) to about 80% (w/w);
(c) PEG 1500 from about 2% (w/w) to about 7% (w/w); and
(d) Kollidon CL from about 5% (w/w) to about 15% (w/w).

Another embodiment provides the pharmaceutical composition, wherein the composition comprises:
(a) 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt 15% (w/w);
(b) Kollidon VA64 77% (w/w);
(c) PEG 1500 5% (w/w); and
(d) Kollidon CL 10% (w/w).

Another embodiment provides the pharmaceutical composition, wherein the composition is a tablet dosage form or a capsule dosage form. Another embodiment provides the pharmaceutical composition, wherein the composition exhibits long term stability.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a method of treating a cancer in a patient in need thereof, comprising administering to the patient a pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been has been subjected to milling.

One embodiment provides a method of treating a cancer in a patient in need thereof, comprising administering to the patient a therapeutically effective amount of a pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to milling.

Another embodiment provides the method, wherein the cancer is selected from a hematologic or a solid malignancy.

One embodiment provides a method of treating a cancer in a patient in need thereof, comprising administering to the patient a pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to lyophilization, or spray drying, or a combination thereof.

One embodiment provides a method of treating a cancer in a patient in need thereof, comprising administering to the patient a therapeutically effective amount of a pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to lyophilization, or spray drying, or a combination thereof.

Another embodiment provides the method, wherein the cancer is selected from a hematologic or a solid malignancy.

### EXAMPLES

The present disclosure is further illustrated by the following examples, which should not be construed as limiting in any way. The experimental procedures to generate the data shown are discussed in more detail below. The disclosure has been described in an illustrative manner, and it is to be understood that the terminology used is intended to be in the nature of description rather than of limitation.

It has been challenging and difficult to develop a stable, non-toxic formulation of Compound 1 lysine salt. Numerous formulations were developed and tested; however, the majority had poor bioavailability and/or proved unstable upon storage, and/or turned to be highly toxic. It has been unexpectedly and surprisingly discovered that the formulations disclosed herein demonstrated good bioavailability in the animal PK studies.

### Example 1: Nanosuspension by Nano-Milling

Nano-milling reduces particle size of the API to nanometer. The initial particle size prior to milling is ˜6-15 micrometer in length.

### Nano-Milling Procedure

Instrument:

| Name | Model | Manufacturer |
|---|---|---|
| Ball mill | MiniCer | Netzsch |

Compounding table for nano-milling slurry

| Component | F14 |
|---|---|
| Compound 1 Lysine salt (API) | 2.0 g |
| HPC (Hydroxypropyl cellulose) | 2.0 g |
| DI-water | QS to 200 mL |

### Procedure:

### Slurry Preparation for Milling:

1. 0.25 L of 1% HPC solution in DI water was prepared.
2. 2.0 g of API was weighed in a beaker and 1% HPC was added up to 200 g to obtain a final concentration of 10 mg/mL in the slurry. Slurry contents was stirred to mix well.

### Ball-Milling Process:

1. Milling beads (140 mL of 0.4 mm zirconium oxide beads) were added to the grinding tank of the mill using a feed funnel.
2. The grinding tank was closed, and mill was set to horizontal operating position.
3. The milling slurry was added to the collecting vessel of the mill.
4. The overhead stirrer was turned on.
5. Cooling water cocks were opened, and the product pump was turned on starting with lowest flow rate.
6. When the product exits the outlet into the collecting vessel, the mill (Netzsch MiniCer Bead Mill) was started at its lowest speed (10 Hz or 600 RPM) and then increased to 2400 RPM in less than a minute.
7. Slurry was milled in the Netzsch MiniCer at 2400 RPM.
8. The milling slurry circulation rate was gradually increased.
9. Samples are collected intermittently, and the milling process was stopped and considered complete when particle size is no longer decreasing per PSD results (Max milling time observed was ˜20 minutes).
10. Final formulation was prepared as described in Table 1A by supplementing PEG 400 and Sodium Benzoate to the nano-milled material and mixed well.

TABLE 1A

| Component | F14_2% w/w |
|---|---|
| Compound 1 Lysine salt (API) | 0.17% |
| HPC (Hydroxypropyl cellulose) | 0.91% |
| DI-water | 90.49% |
| PEG 400 | 8.32% |
| Sodium benzoate | 0.10% |

Results: Particle size distribution as determined by dynamic light scattering of Compound 1 lysine salt nanosuspension post milling is shown below in Table 1B. Samples for particle size distribution was prepared by diluting the Nano milled sample by 100-fold with Di-water. Zetasizer was utilized to measure particle size distribution. PdI is the representation of the distribution of size populations within a given sample. This is a numerical range from 0.0 (for a perfectly uniform sample) through 1.0 (for a highly polydisperse sample with multiple particle size populations). The value D10 refers to a diameter of particles in the sample wherein 10% of the particles in the sample have a diameter smaller than this value (118.8 nm). The value D50 refers to a diameter of particles in the sample wherein 50% of the particles have a diameter smaller than this value (249.0 nm). The value D90 refers to a diameter of particles in the sample wherein 90% of the particles in the sample have a diameter smaller than this value (611 nm).

| | |
|---|---|
| Z-average | Particle size distribution (PSD) in nm |

| (nm) | PdI | D10, v | D50, v | D90, v |
|---|---|---|---|---|
| 212.7 | 0.265 | 118.8 | 249.0 | 611 |

The composition from milling is a uniform, yellow and translucent suspension. A stable suspension was observed during the storage (at 2-8° C.) with no particle aggregation or precipitation observed. Particle size of API was reduced to 212 nm by ball-mill technology using specific stabilizing agents, and thus generated a uniform nanosuspension.

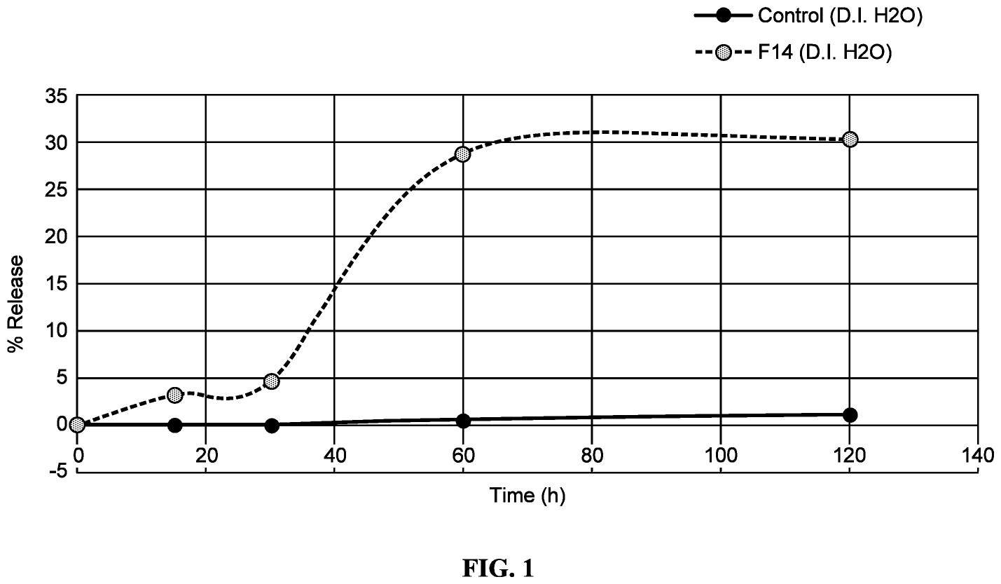

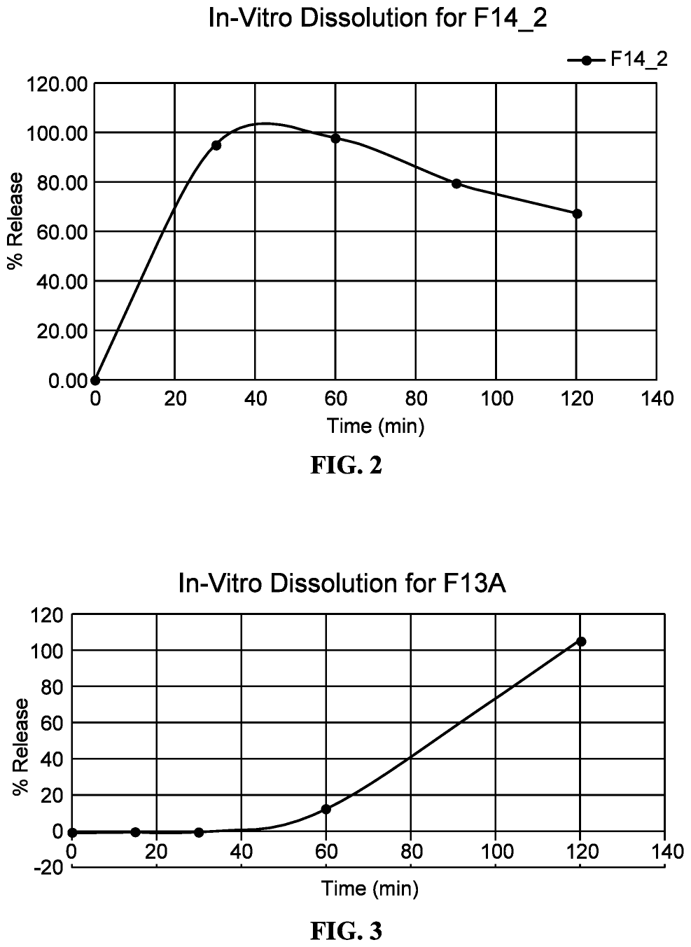

In vitro dissolution testing of the nano milled material prior to supplementing with PEG400 was studied and the result is provided in FIG. 1. The in vitro dissolution testing of nano milled material post supplementing with PEG 400 was studied and result is provided in FIG. 2. The comparison of dissolution profile of nanomilled material prior to and post supplementation with PEG 400 demonstrated significant increase in the dissolution of Compound 1 Lysine salt with an in vitro dissolution threshold reaching a max of 30% within 60 min for the nanomilled material prior to supplementing with PEG 400 versus 100% release within 40 minutes for the nanomilled material upon supplementation of PEG 400.

The stability of the nanosuspension based on particle size distribution under refrigerated condition and room condition is shown below in Table 2.

TABLE 2

| Storage | Time | Z-average | |
|---|---|---|---|
| condition (° C) | point—day(s) | (nm) | PdI |
| Initial | T-0 | 212.7 | 0.265 |
| 2-8 | 4 | 254.3 | 0.364 |
| | 11 | 266.7 | 0.356 |
| | 21 | 276.7 | 0.392 |
| 25 | 11 | 251.1 | 0.318 |

Assay and impurity testing for the nanosuspension under stability testing conditions is presented below in Table 3.

TABLE 3

| Storage | Time | Assay | % Recovery over |
|---|---|---|---|
| condition (° C) | point—day(s) | (mg/g) | T = 0 |
| Initial | 0 | 9.60 | N/A |
| 2-8 | 33 | 9.63 | 100.2 |
| 25 | 33 | 9.51 | 99.0 |

Particulate growth was observed at room temperature storage conditions. This is possibly due to the phenomenon of Ostwald ripening. No particulate growth through time was observed under 2-8° C. test conditions.

The suspension formulations were found chemically stable across both the storage conditions.

For the ease of administration of this formulation, the nanosuspension can be subjected to spray drying to convert it into a fine powder that can be filled into a capsule or compressed into tablets and administered orally. Additionally, when the nanosuspension is dried via spray drying, there is no concern of particulate growth or Ostwald ripening since there is no moisture to promote the same.

### Example 2: Lyophilized Formulation

The use of surfactants and disintegrants was studied to determine the improvement of bioavailability and dissolution rate.

Instrument:

| Name | Mfg | Model |
|---|---|---|
| Lyophilizer (Dura-Stop | FTS Systems | TDS3C0T5100 |
| Microprocessor Control | | |
| Stoppering Tray Dryer) | | |

Materials:

| Name | Function | Mfg |
|---|---|---|
| Compound 1 | Active | Tachyon |
| Lysine Salt | ingredient | |
| Kollidan VA64 | Binding agent | BASF |
| PEG 1500 | Solubilizing | Spectrum |
| | agent | |
| Kollidon CL | Disintegrant | BASF |

TABLE 4

| Component | F13A % (w/w) |
|---|---|
| Compound 1 Lysine Salt | 15 |
| Kollidon VA64 | 77 |
| PEG 1500 | 5 |
| Kollidon CL | 3 |

### Procedure:

1. Appropriate quantities of API, Kollidan VA64 and PEG 1500 are weighed into a lyophilization tube.
2. DI-water was added to dissolve the powder, in certain cases a combination of Ethanol and DI-water would be utilized for better solubilization. This was followed by sealing and mixing.
3. The above solution was transferred into a glass container and was placed in −40° C. freezer until all solutions are solid
4. Lyophilized at −40° C. for overnight.
5. Solid was pulverized with spatula and mixed with the required quantity of Kollidon CL
6. Appropriate quantity of formulated bulk is filled into capsule (Mfg: Capsulgel, Size: 00, Lot #7171508[2]) to attain the desired drug load for oral administration in dog PK studies. Capsule size 000 size 000 capsules may be used to attain the desired drug load.
7. Stability assay and dissolution testing were performed, and the results provided in Table 5 and FIG. 3 respectively. Conditions and procedure for dissolution are provided in example 3.
8. The formulation monitored for stability under controlled ambient conditions. No degradation observed as indicated in Table 5.

TABLE 5

| Storage | Time point | Assay | % Total |
|---|---|---|---|
| condition (° C) | (months) | purity (%) | impurities |
| 25 | 0 | 93.0 | 0.35 |
| | 1 | 96.0 | 0.27 |

### Example 3: Additional Formulation Studies

### Hot Melt Formulations

Materials used for hot melt compositions are provided in Table 6. The components of each hot melt composition are provided in Table 7.

TABLE 6

| Name | Mfg |
|---|---|
| Compound 1 Lysine Salt | Tachyon |
| Kollidon VA64 | BASF |
| Eudragit EPO | Evonik |
| Kollidon 12 PF | BASF |
| Poloxamer 188 | Spectrum |
| | chemicals |
| PB80 (Polysorbate 80) | |

TABLE 7

| | F1 | F7 | F9 | F10 | F11 | F12 |
|---|---|---|---|---|---|---|
| Component | % (w/w) | % (w/w) | % (w/w) | % (w/w) | % (w/w) | %(w/w) |
| Compound 1 Lysine Salt | 8.33 | 8.33 | 8.33 | 8.33 | 8.33 | 8.33 |
| Kollidon VA64 | 91.67 | | | | | |
| Eudragit EPO | | 91.67 | | | | |
| Kollidon 12 PF | | | 91.67 | | 88.67 | 85.67 |
| Poloxamer 188 | | | | 91.67 | | |
| PB80 (Polysorbate 80) | | | | | 3 | 6 |

### Procedure:

1. The oven was pre-heated to 160° C.
2. The ingredients were weighed into a mortar.
3. The components were mixed thoroughly with a pestle.
4. The samples were placed into the pre-heated oven for 5 min. If the samples did not melt, then an additional 5 min of heat was applied
5. Samples were cooled down to room temperature.
6. Each of the formulation was then jet milled to obtain fine granules.

No impurity or degradation was observed from purity assessment of each of the formulations from the hot melt experiments in comparison to the purity of active ingredient prior to formulation indicating that the active ingredient is stable under high temperature utilized in preparations of these hot melt formulations. The dissolution profiles for these formulations were evaluated per procedure outlined.

Dissolution and assay testing for API, F1, F7, F9, F10, F11 and F12.

| | |
|---|---|
| System | HP Agilent 1100 |
| Apparatus | USP Apparatus 1 (Basket) |
| Temperature | 37.0 ± 0.5° C. |
| Rotation Speed | 100 rpm |
| Medium | DI water |
| Medium Volume | 500 |
| Sampling Time Point | 5, 15, 30, 60 and 120 minutes |
| Sampling Volume | 1 mL |
| Sample Filter | 35 μm, LabECX, Product # SF-17-4010 |

### Sink Condition:

1. 5 mg of API was placed into a glass vial.
2. Added 0.2 mL of diluent (DI water and SGF) into the vial, then sonicate and vortex to dissolve the API.
3. If the solution is not clear after 5 minutes, add additional 0.2 mL of diluent into the vial, then sonicate and vortex to dissolve the API.
4. Repeat step 4 till all API is dissolved.
5. Calculate the final concentration of API and the maximum dissolvable dose used in dissolution test.

### Dissolution Testing:

6. Transfer 500 mL of DI water dissolution medium into each vessel.
7. Equilibrate the dissolution medium to 37.0±0.5° C.
8. Transfer 600 mg of each formulation granule into a capsule.
9. Transfer a capsule into a basket and lower to the position. Start stirring at 100 RPM.
10. At the stated time(s), sample 1 mL solutions by auto-sampler.
   Assay Sample Preparation (0.3 mg/mL):
11. Weigh out 18 mg of granules into a 5-mL volumetric flask.
12. Dissolve and dilute up to the volume with Diluent (50% ACN in H2O).
13. Mix well.

TABLE 8

| | Compound 1 | Compound 1 |
|---|---|---|
| Name | in H₂O | in SGF |
| Diluent | DI water | SGF |
| Final Concentration, mg/mL | 4.3 | 3.7 |
| Maximum dissolvable | 717 | 617 |
| dose used in | | |
| dissolution test, mg | | |

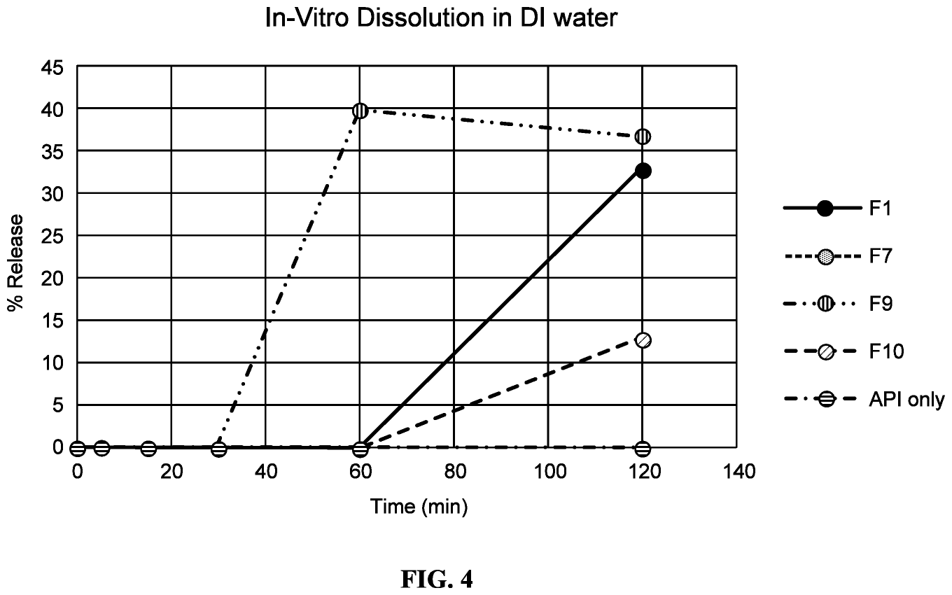

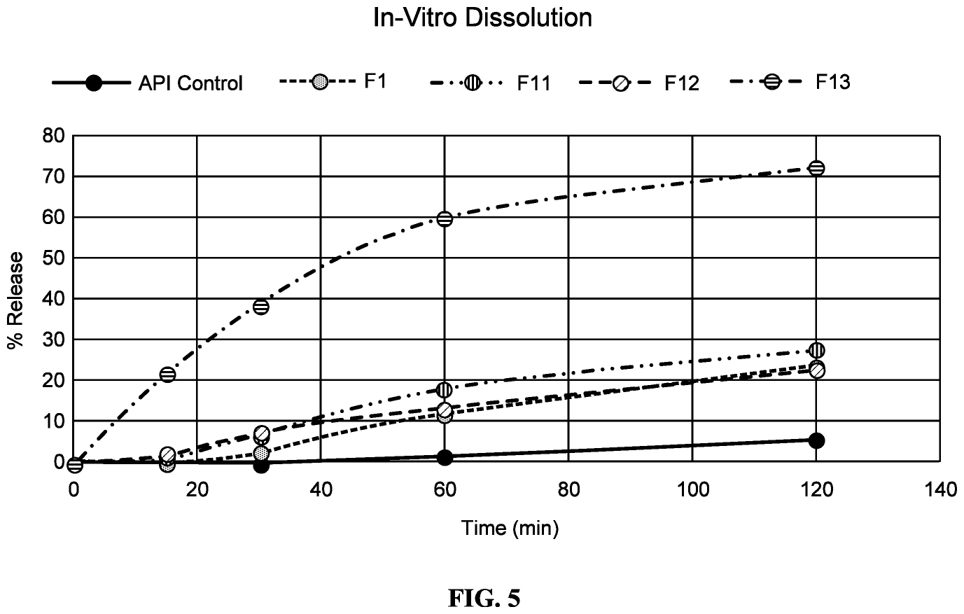

Conclusions: Dissolution profile in DI water are provided in FIG. 4. API only and F7 showed no release at all after 120 minutes from the dissolution study. In contrast, F1, F9 and F10 showed faster dissolution rate. Amongst them F9 showed about 40% release after 1 hour. Dissolution results for formulation F11 and F12 are provided in FIG. 5. Preparations F11 and F12 were jet-milled, and the granule particle size was about 5 micron, however no observed improvement in the dissolution rate was observed. F11 and F12 contains different amount of Polysorbate 80 (PB80), and the result suggested that it has no impact on dissolution rate

### Lyophilized Formulations

Purpose: To prepare a fast-dissolving formulation by freeze drying.

TABLE 9

| Component | F13 % (w/w) |
|---|---|
| Compound 1 Lysine Salt | 10 |
| Kollidan VA64 | 90 |

### Procedure:

1. Compound 1 Lysine Salt and Kollidan VA64 were weighed into an appropriate glass vial.
2. tert-butanol was added to dissolve the powder, sealed, and then mixed; the mixture was heated if necessary.
3. The vial was placed in a −20° C. freezer until all solutions are solid.
4. Vial was lyophilized at −20° C. for 16 hr and at 30° C. for 3-5 hr.
5. Final solid was pulverized with spatula.

Results: An off-white fluffy powder was obtained. Powder was filled into HPMC capsules and subjected to dissolution test. Formulation F13 provided improved dissolution rate and percentage. Data are provided in FIG. 5.

### Clear Sol and SEDDS Technology

Purpose: To screen for two additional compositions for improving the oral availability of Compound 1 lysine salt. ClearSol technology (F15) is an FDA approved lipid-based proprietary solubilizer described in US patent application publication 2020/0368159A1. Self-emulsifying drug delivery system (SEDDS) (F16 and F17) were also investigated.

Materials:

| Name | Mfg |
|---|---|
| Compound 1 Lysine Salt | Regis |
| ClearSol | LPI |
| SEDDS vehicle 1 | LPI |
| SEDDS vehicle 2 | LPI |

TABLE 10

| | SEDDS V1 (F16) | SEDDS V2 (F17) |
|---|---|---|
| Components | % (w/w) | % (w/w) |
| Miglyol 812N | 44.8 | |
| Capmul MCM | 25.4 | 36.84 |
| PL90G | | 42.1 |
| Polysorbate 80 (PB80) | 10 | |
| Vitamin E TPGS | | 10.53 |
| Triethyl Citrate | 19.8 | |
| PEG400 | | 10.53 |
| Total | 100 | 100 |

TABLE 11

| | F15 | F16 | F17 |
|---|---|---|---|
| Component | % (w/w) | % (w/w) | % (w/w) |
| Compound 1 Lysine | 1 | 1 | 1 |
| Salt | | | |
| ClearSol | 99 | | |
| SEDDS vehicle 1 | | 99 | |
| SEDDS vehicle 2 | | | 99 |

### Procedure:

1. Compound 1 Lysine Salt was weighed into an appropriate containers/vials
2. Vehicle was added to each vial to achieve the total weight of 2 g
3. The contents were Vortexed and sonicated to dissolve the API.

Results: The active ingredient was not completely miscible in the formulations F15, F16 or F17. Therefore, formulation containing Clear Sol and SEDDS-2 formulations were not further studied. The SEDDS-1 formulation was further studied with the addition of co-solvents.

### Modified Clear Sol and SEDDS Technology

Purpose: To prepare F19 (Compound 1 Lysine salt in self-emulsifying vehicle), 10% Propylene Glycol (PG) in SEDDS V1.

Materials:

| Name | Mfg | Grade | Lot # |
|---|---|---|---|
| Compound 1 Lysine Salt | Regis | N/A | R23912-002-2 |
| SEDDS vehicle 1 | LPI | N/A | To be filled |

TABLE 12

| | SEDDS V1 | 10% PG in SEDDS V1 |
|---|---|---|
| Components | (F16) | (F19) |
| Propylene glycol (PG) | | 10 |
| SEDDS V1 | | 90 |
| Miglyol 812N | 44.8 | |
| Capmul MCM | 25.4 | |
| Polysorbate 80 (PB80) | 10 | |
| Triethyl Citrate | 19.8 | |
| Total | 100 | 100 |

TABLE 13

| | F19 |
|---|---|
| Component | % (w/w) |
| Compound 1 Lysine Salt | 0.2 |
| 10% PG in SEDDS V1 | 99.8 |

### Procedure:

Vehicle preparation: 10% PG in SEDDS V1 (2.7 g SEDDS V1 was mixed with 0.3 g PG).
1. 2 mg of Compound 1 Lysine Salt was weighed in an appropriate container.
2. Vehicle was then added to the vial according to the compounding table to achieve the total weight of 1 g.
3. The contents were vortexed and sonicated to dissolve the API.
4. The formulation was then subjected to dissolution testing.

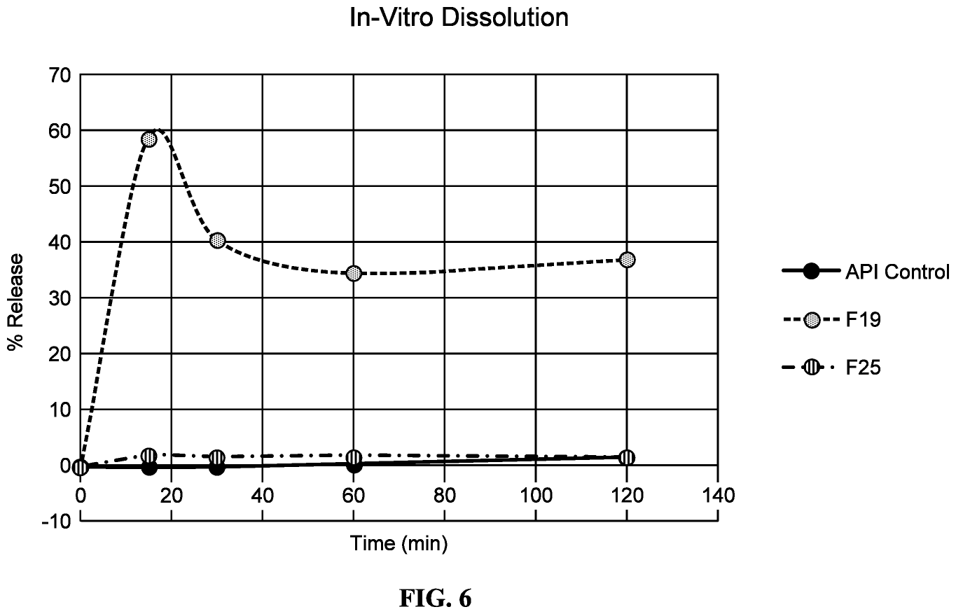

Results: Clear solution was observed indicating active ingredient was completely miscible in the vehicle. Formulation 19 demonstrated less % release in comparison to F14_2 or F13A, see FIG. 6. Further modification of Formulation F19 through pH adjustment was performed to determine if the dissolution could be increased further.

### pH Modified SEDDS Formulation

Purpose: To optimize F19 (SEDDS formulation) by increasing the pH with ammonium hydroxide to improve the release.

Materials:

| Name | Mfg | Grade | Lot # |
|---|---|---|---|
| F19 | LPI | N/A | 390-1-61 |
| Ammonium hydroxide (28-30%) | Fisher Chemical | ACS | 170719 |

TABLE 14

| | F19C |
|---|---|
| Component | % (w/w) |
| Compound 1 Lysine Salt | 0.16 |
| Propylene glycol (PG) | 9.90 |
| Miglyol 814N | 39.94 |
| Capmul MCM | 22.64 |
| PB80 | 8.91 |
| Triethyl Citrate | 17.65 |
| *Ammonium hydroxide (28-30%) | 0.79 |

*Equal to 0.24% ammonium hydroxide in formulation.

TABLE 15

| | F19C |
|---|---|
| Component | (g/400 g) |
| Compound 1 Lysine Salt | 0.63 |
| Propylene glycol (PG) | 39.62 |
| Miglyol 814N | 159.74 |
| Capmul MCM | 90.57 |
| PB80 | 35.66 |
| Triethyl Citrate | 70.60 |
| Ammonium hydroxide (28-30%) | 3.18 |

### Procedure:

1. 400 mL of Formulation F19 was mixed with 3.2 mL ammonium hydroxide (28-30%).
2. Assay and dissolution testing was performed on the pH-adjusted formulation

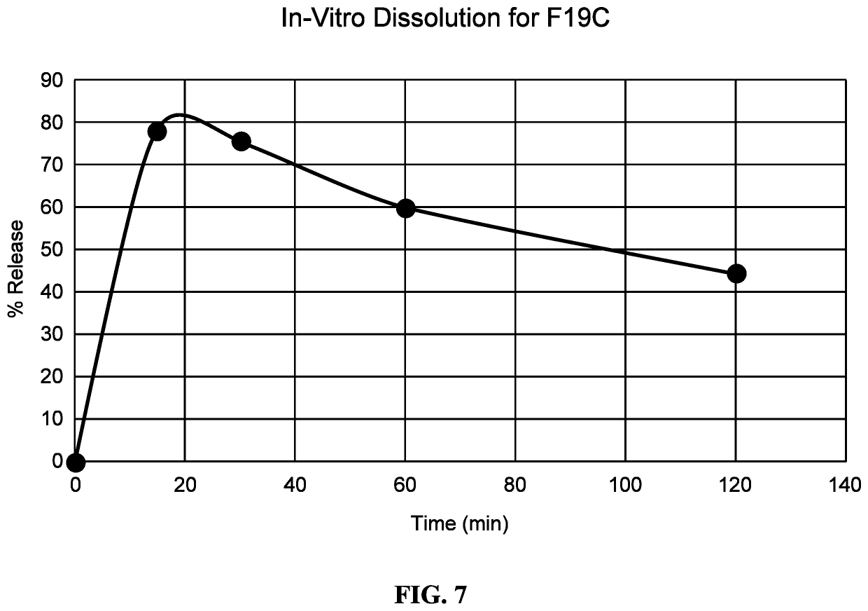

Results: The dissolution data is provided in FIG. 7. Compared to F19, F19C showed improvement in the initial release from 60 to 80%. Re-equilibrium after 15 minutes was observed. However, the released drug may be absorbed by biological system (in vivo study) and may not observe the re-equilibrium issue in vivo. Since this formulation showed good dissolution; this formulation was subjected to another Dog PK study along with two other formulations F13A and F14_2 (which were then concluded as the lead formulations) for comparison of Bioavailability.

ClearSol Technology with Nanomilled Material

Purpose: To evaluate if formulation F14 (see example 1) could be further optimized by incorporating the Clearsol solubilizer to the nano-milled material.

Materials:

| Name | Mfg |
|---|---|
| Compound 1 Lysine Salt | Regis |
| F14 | LPI |
| ClearSol (F101V) | LPI |

TABLE 16

| | F25 |
|---|---|
| Component | % (w/w) |
| F14 | 16 |
| ClearSol | 68 |
| DI-water | 16 |

### Procedure:

1. The components are weighed into the vial according to the compounding table.
2. The contents was then vortexed and sonicated.

Results: The dissolution data is provided in FIG. 6. For Formulation F25, there was no release upon dissolution. Therefore, formulation F25 was not studied further.

### Example 4: Pharmacokinetic and Bioavailability Study

A summary of the dog pharmacokinetic studies performed with formulations prepared in Examples 1-3 above is provided in Table 17.

TABLE 17

| | | PEG/MC | | | | |
|---|---|---|---|---|---|---|
| PK parameter | IV PK | (Internal Control) | F13^{#} | F19C^{$} | F14_2^{$} | F13A^{$} |
| Formulation | 10% propylene | Polyethylene Glycol | Kollidon | SEDDS | Nano-milled | Kollidon |
| type | glycol pH | (PEG)/Methylcellulose | (without | | formulation | (+PEG1500) |
| | adjusted to | (MC) Composition: | PEG1500) | | supplemented | |
| | alkaline pH | 10% PEG 400 and | | | with PEG 400 | |
| | | 90% of 0.5% MC | | | | |
| Dose (mg/kg) | 1 IV | 8 PO | 2.6* Capsule/PO | 6 PO | 6 PO | 5.9* Capsule/PO |
| Route^{&&} | | | | | | |
| C₀ (ng/ml) | 3,615 ± 750 | Not applicable | Not applicable | Not applicable | Not applicable | Not applicable |
| Cₘₐₓ (ng/ml) | Not applicable | 5,280 ± 2,613 | 738 | 394 ± 271 | 3,753 ± 1526 | 2,917 ± 2335 |
| | for IV | | | | | |
| AUC_{0-inf} | 2526 ± 509 | 15,337 ± 6,787 | 4011 | 3345 ± 4,597 | 12430 ± 3883 | 10,732 ± 8,715 |
| (ng · h/mL) | | | | | | |
| Tₘₐₓ (hr) | Not applicable | 1.0 | 2 | 1.5 ± 0.87 | 0.83 ± 0.29 | 1.17 ± 0.764 |
| | for IV | | | | | |
| T_{1/2} (hr) | 3.90 ± 0.628 | 3.59 ± 0.8 | 5.71 | 3.43 ± 1.43 | 2.97 ± 0.56 | 5.12 ± 1.80 |
| Cl (ml/min/kg) | 6.79 ± 1.43 | Not applicable | Not applicable | Not applicable | Not applicable | Not applicable |
| Vss (L/kg) | 1.12 ± 0.482 | Not applicable | Not applicable | Not applicable | Not applicable | Not applicable |
| % F | 100 | 81 | 48 | 27.7 | 87.4 | 81.8 |

*Indicates the formulation was filled into Gelatin capsules and administered orally
^{#}Relative bioavailability comparison based on data from PEG/MC oral formulation
^{$}Absolute bioavailability (% F) is the dose-corrected area in comparison IV formulation data listed in this table
^{&&}Dose calculated based on free acid equivalent of Compound 1 Lysine salt

Results: Relative bioavailability (% F) for F13 was calculated by comparing the exposure of F13 to the exposure of the internal control PK using PEG/MC (orally administered).

Based on the above results, the study with F13 indicated only ˜48% bioavailability in comparison to the PEG/MC as the control. Therefore, this formulation was further optimized to F13A via addition of solubilizing agents such as PEG which in turn helped improve the oral bioavailability significantly (82% in comparison to IV). Likewise, good oral bioavailability was observed for Formulation F14_2 (87%) which is a nanomilled API with HPC and PEG 400 excipients.

The Kollidon-based formulation such as F13 and F13A showed a lower Cₘₐₓ and longer T_{1/2}. These are preferred pharmacokinetic parameters as higher Cₘₐₓ increases probabilities of interacting with off-targets. As a potent KMD4 inhibitor, it may not be required to have a high Cₘₐₓ to inhibit KDM4, but longer T_{1/2} may increase duration of target interaction.

Formulation F19C demonstrated good in vitro dissolution, however poor bioavailability was observed from the dog PK study

The high bioavailability of formulations F13A and F14_2 in the dog PK study these two are the lead formulations for clinical development.

A rat pharmacokinetic study was performed with formulation F13A. The data is provided in Table 18.

TABLE 18

| PK parameter | Rat iv-TACH | Rat-po-TACH |
|---|---|---|
| Formulation | 10% propylene glycol in | F13A-powder |
| | water | Not capsule |
| | pH adjusted to alkaline | |
| | pH | |
| Dose (mg/kg) | 2.5 | 8 |
| Route | IV | PO |
| C0 (ng/ml) | 8,781 ± 1,267 | Not applicable |
| Cₘₐₓ (ng/ml) | Not applicable for IV | 1,933 ± 254 |
| AUC0-t (ng*hr/ml) | 3,755 ± 262 | 8,603 ± 1,511 |
| AUC0-inf (ng*hr/ml) | 3,763 ± 264 | 8,625 ± 1,505 |
| Tmax (hr) | Not applicable for IV | 2.3 ± 1.53 |
| t_{1/2} (hr) | 1.88 ± 0.21 | 2.5 ± 0.65 |
| Cl (ml/min/kg) | 11.1 ± 0.77 | Not applicable |
| Vss (L/kg) | 0.695 ± 0.10 | Not applicable |
| Bioavailability (% F) | 100 | 71.6 |

Results: F13A was solubilized in water and dosed with F13A solution. High bioavailability was confirmed in rats. The absolute dose adjusted bioavailability was calculated based on the IV data generated in rats.

### Example 5: Spray Drying Process

The formulation F13A was further optimized for production on a larger scale. An improved process utilizes spray drying followed by lyophilization (for extended drying) instead of lyophilization throughout (as described above). The spray drying process followed by lyophilization improved the procedure of removing the processing solvents (ethyl alcohol and water) from the formulation to acceptable limits. Whereas the initial process of freezing a solution, lyophilizing the solution overnight, then pulverizing the resulting product is not the most optimal process for commercial scale production. Therefore, the spray drying process followed by lyophilization (for extended drying) was optimized for larger scale production of this formulation.

Purpose: To develop a Spray-drying process.

Instruments:

| Name | Mfg | Model |
|---|---|---|
| Spray Dryer | Yamato | GS310 |
| Pump | Thomson | 1212100 |

Materials:

| Name | Mfg |
|---|---|
| Compound 1 Lysine | Regis |
| Salt | |
| Kollidan VA64 | BASF |
| PEG 1500 | Spectrum |
| Kollidon CL | BASF |
| Ethanol, 200 proof | Decon |
| (EtOH) | |

TABLE 19

| | F13A |
|---|---|
| Component | % (w/w) |
| Compound 1 Lysine Salt | 15 |
| Kollidon VA64 | 77 |
| PEG 1500 | 5 |
| Kollidon CL | 3 |

TABLE 20

| | F13A |
|---|---|
| Component | (g/16 g) |
| Compound 1 Lysine Salt | 2.4 |
| Kollidon VA64 | 12.3 |
| PEG 1500 | 0.8 |
| Kollidon CL | 0.5 |

### Procedure for Spray Drying Solvent Evaluation:

The fast-dissolving freeze dried/lyophilization formulation (15% Compound 1 lysine salt, 77% Kollidon VA64, and 5% Polyethylene Glycol 1500), were mixed with various diluents and evaluated for the solution appearance and the amount of formulation that was dissolved. The results are provided in Table 21

TABLE 21

| | | Concentration of |
|---|---|---|
| | | active ingredient |
| Solvent | Appearance | (g/100 mL) |
| 80% Ethanol | Clear | 7.8 |
| 100% Isopropyl Alcohol | Cloudy | 1.0 |
| 80% Isopropyl Alcohol | Clear | 7.5 |
| 50% Isopropyl Alcohol | Powder not dissolved (non- | 3.0 |
| | homogenous) | |
| 20% Isopropyl Alcohol | Cloudy | 1.0 |
| 80% Methanol | Cloudy | 3.3 |
| 80% Acetonitrile | Cloudy | 2.1 |

Results: Based on the appearance and greatest concentration results from the solvent evaluation, the 80% ethanol solution was chosen as the lead spray-drying solvent.

After the 80% Ethanol solution was chosen as the lead spray-drying solvent. The spray drying process was further optimized through the evaluation of the inlet temperature, spray pressure, orifice pressure, and pump rate. Five additional (5) spray drying conditions were evaluated based on % assay, % recovery, and appearance. The spray drying conditions are provided in Table 22. The promising conditions from the preliminary evaluation is presented in Table 23 for comparison.

### Procedure for Spray Drying Process Optimization:

The spray drying conditions listed in Table 22 were evaluated.

TABLE 22

| Parameter | Condition 2 | | Condition 3 | | Condition 4 | | Condition 5 | | Condition 6 | |
|---|---|---|---|---|---|---|---|---|---|---|
| Inlet Temperature | 80° | C. | 90° | C. | 60° | C. | 100° | C. | 100° | C. |
| Spray Pressure | 0.1 | MPa | 0.1 | MPa | 0.1 | MPa | 0.1 | MPa | 0.1 | MPa |
| Orifice Pressure | 0.5 | KPa | 0.3 | KPa | 0.25 | KPa | 0.25 | KPa | 0.25 | KPa |

| | | | | | |
|---|---|---|---|---|---|
| Pump Rate | 2 | 3 | 3 | 3 | 5 |

TABLE 23

| | | Theoretical | | |
|---|---|---|---|---|
| | Assay | Assay | % | |
| Condition | (%, w/w) | (%, wt/wt) | Recovery | Appearance |
| Condition 3 | 15.1 | 15.5 | 97.76 | White to off white |
| | | | | loose powder |
| Condition 5 | 16.4 | 16.7 | 98.13 | White to off white |
| | | | | loose powder |
| Condition 6 | 15.4 | 15.5 | 99.73 | White to off white |
| | | | | loose powder |

Extended drying (Lyophilization) of spray dried sample was performed at 40° C. preheated trays. The spray-dried material is then weighed and transferred to the preheated trays. The vacuum was set to 40-150 mTorr and the material was dried for a minimum of 12 hours. After 12 hours, the shelf temperature was then reduced to 25° C. and subjected to further drying for at least 1 hour.

The formulation was monitored for stability under controlled ambient conditions. No degradation observed as indicated in Table 24.

TABLE 24

| Storage | Time point | Assay range 90-110% | % Total |
|---|---|---|---|
| condition (° C.) | (months) | (% recovery against T0) | impurities |
| 25 | 0 | N/A | 0.35 |
| | 1 | 104 | 0.27 |
| | 3 | 99 | 0.31 |

### Example 6: Evaluation of Compound 1 in Cancer Cell Line Panel

In a larger screening study, compound 1 was evaluated across a broad cancer cell line panel (OncoPanel; HD Biosciences) composed of 301 cancer cell lines from different tumor types.

2D cell viability inhibition assays were performed in multiwell plates seeded with cells from the panel. After seeding, the plates were cultured overnight in a humidified incubator at 37° C. to promote adherence. Assays were initiated in individual wells by adding either DMSO as a vehicle control, growth media as a blank, or serially diluted compound 1 (10 μM to 0.0005 μM with 1:3 serial dilution). Cultures were incubated for 168 hours after which the number of viable cells in each test well was assessed using the CellTiter-Glo® Luminescent Cell Viability Assay. Luminescence readouts were performed using an EnVision® Multilabel Reader and the compound 1 readouts were normalized to the DMSO control readouts, expressed as a percent of the control. Outliers were flagged out by visual inspection. Percent of control was plotted against the corresponding compound 1 concentration and the absolute IC50 value was determined using four-parameter logistic non-linear regression as the concentration where inhibition was 50% of the control. For maximum inhibition <50%, the absolute IC50 was reported as >10. In addition to EC50 and IC50, AUC was determined and was normalized to the area corresponding to theoretical no-inhibition (the rectangular area defined by the compound dose range and 0 to 100 in y). Emax was recorded as the minimum in y with the compound dose range. For those cell lines where Emax>40, EC50 was manually set to 10 μM. A hierarchical clustering algorithm based on Euclidean distance between the standardized input values and “Ward.D” method as implemented in R hclust function was used to visualize the compound 1 cellular potency data. The sensitive/moderate/resistant calls for each cell line was determined by visually inspecting the resulting clustering dendrogram. All the analyses were done in R Core Team.

Results demonstrated that compound 1 potently inhibited proliferation of cancer cell lines and displayed a highly selective potency profile with absolute IC₅₀ values ranging from <500 pM to >10 μM. Among the 301 cell lines, 209 were inhibited by compound 1 with IC₅₀ values <1 μM while 89 lines were insensitive with IC₅₀ values >10 μM. Table 25 provides the results for all 301 cell lines.

TABLE 25

| Cell line | IC₅₀(μM) | EC₅₀(μM) | AUC | Emax | Tumor Type | Tumor Subtype |
|---|---|---|---|---|---|---|
| NCIH1618 | 5.00E−04 | 0.000508 | 0.022662 | 0.488613 | lung | small cell carcinoma |
| NCIH1048 | 0.000706 | 0.000508 | 0.147882 | 7.150228 | lung | small cell carcinoma |
| NCIH1155 | 0.000765 | 0.000884 | 0.075713 | 0.26377 | lung | large cell carcinoma |
| NCIH1666 | 0.001165 | 0.000508 | 0.298302 | 21.65869 | lung | bronchioloalveolar |
| | | | | | | adenocarcinoma |
| REH | 0.00121 | 0.001324 | 0.095695 | 0.233625 | haematopoietic | acute lymphoblastic B cell |
| | | | | | and lymphoid | leukaemia |
| | | | | | tissue | |
| MOLT4 | 0.00125 | 0.000508 | 0.137568 | 0.604781 | haematopoietic | acute lymphoblastic T cell |
| | | | | | and lymphoid | leukaemia |
| | | | | | tissue | |
| NCIH209 | 0.001307 | 0.00148 | 0.186896 | 8.610089 | lung | small cell carcinoma |
| JURKAT | 0.001488 | 0.001655 | 0.117145 | 0.211954 | haematopoietic | acute lymphoblastic T cell |
| | | | | | and lymphoid | leukaemia |
| | | | | | tissue | |
| NCIH1694 | 0.001753 | 0.000508 | 0.321443 | 21.59348 | lung | small cell carcinoma |
| HCT15 | 0.001791 | 0.001473 | 0.183818 | 4.540784 | large intestine | adenocarcinoma |
| JEKO1 | 0.001804 | 0.002309 | 0.117241 | 0.087378 | haematopoietic | mantle cell lymphoma |
| | | | | | and lymphoid | |
| | | | | | tissue | |
| TALL1 | 0.001805 | 0.000895 | 0.199872 | 4.509292 | haematopoietic | acute lymphoblastic T cell |
| | | | | | and lymphoid | leukaemia |
| | | | | | tissue | |
| A2780 | 0.002137 | 0.002099 | 0.181084 | 3.567055 | ovary | adenocarcinoma |
| LOUCY | 0.002474 | 0.002471 | 0.188038 | 2.522241 | haematopoietic | acute lymphoblastic T cell |
| | | | | | and lymphoid | leukaemia |
| | | | | | tissue | |
| DLD1 | 0.002533 | 0.001467 | 0.226049 | 5.540107 | large intestine | adenocarcinoma |
| U937 | 0.002554 | 0.002732 | 0.180761 | 1.946235 | haematopoietic | diffuse large B cell |
| | | | | | and lymphoid | lymphoma |
| | | | | | tissue | |
| NCIH2081 | 0.002568 | 0.001459 | 0.304851 | 17.9809 | lung | small cell carcinoma |
| TF1 | 0.002586 | 0.00158 | 0.288323 | 11.54969 | haematopoietic | acute myeloid leukaemia |
| | | | | | and lymphoid | |
| | | | | | tissue | |
| RL | 0.002993 | 0.001877 | 0.236617 | 1.95413 | haematopoietic | B cell lymphoma |
| | | | | | and lymphoid | unspecified |
| | | | | | tissue | |
| NCIH2023 | 0.003031 | 0.002842 | 0.288394 | 13.34318 | lung | adenocarcinoma |
| NAMALWA | 0.003206 | 0.003265 | 0.24309 | 6.45672 | haematopoietic | Burkitt lymphoma |
| | | | | | and lymphoid | |
| | | | | | tissue | |
| TE14 | 0.003393 | 0.002324 | 0.306836 | 12.67238 | oesophagus | squamous cell carcinoma |
| KYSE510 | 0.003395 | 0.003418 | 0.199839 | 0.579743 | oesophagus | squamous cell carcinoma |
| DMS79 | 0.003467 | 0.00128 | 0.384485 | 27.09377 | lung | small cell carcinoma |
| NCIH2286 | 0.003508 | 0.00253 | 0.291713 | 11.19933 | lung | small cell carcinoma |
| NCIH82 | 0.003543 | 0.003315 | 0.250228 | 4.909701 | lung | small cell carcinoma |
| NCIH2087 | 0.003555 | 0.002583 | 0.382152 | 21.97574 | lung | adenocarcinoma |
| PF382 | 0.003685 | 0.001894 | 0.276144 | 8.404679 | haematopoietic | acute lymphoblastic T cell |
| | | | | | and lymphoid | leukaemia |
| | | | | | tissue | |
| YD38 | 0.003771 | 0.003547 | 0.298266 | 13.63878 | upper | squamous cell carcinoma |
| | | | | | aerodigestive tract | |
| HPAC | 0.003928 | 0.002376 | 0.396472 | 20.84918 | pancreas | ductal carcinoma |
| PECAPJ34CLO | 0.003966 | 0.003807 | 0.311032 | 11.80269 | upper | squamous cell carcinoma |
| NEC12 | | | | | aerodigestive tract | |
| MV411 | 0.004017 | 0.003916 | 0.216311 | 0.056467 | haematopoietic | acute myeloid leukaemia |
| | | | | | and lymphoid | |
| | | | | | tissue | |
| AZ521 | 0.004189 | 0.003845 | 0.287487 | 8.891497 | small intestine | NS |
| SNU16 | 0.004217 | 0.003693 | 0.282065 | 7.431244 | stomach | undifferentiated |
| | | | | | | adenocarcinoma |
| TE11 | 0.00428 | 0.003854 | 0.304041 | 10.15582 | oesophagus | squamous cell carcinoma |
| NCIN87 | 0.004292 | 0.003935 | 0.313195 | 10.80159 | stomach | NS |
| P31FUJ | 0.004324 | 0.002434 | 0.285987 | 7.305396 | haematopoietic | acute myeloid leukaemia |
| | | | | | and lymphoid | |
| | | | | | tissue | |
| CORL279 | 0.004345 | 0.004231 | 0.315531 | 11.3227 | lung | small cell carcinoma |
| HUTU80 | 0.004361 | 0.004208 | 0.301592 | 8.150503 | small intestine | adenocarcinoma |
| HCC95 | 0.0044 | 0.003342 | 0.407204 | 17.03582 | lung | squamous cell carcinoma |
| RT11284 | 0.004435 | 0.00414 | 0.371749 | 18.19969 | urinary tract | NS |
| G401 | 0.004449 | 0.004392 | 0.250793 | 3.80796 | soft tissue | NS |
| K562 | 0.004556 | 0.004083 | 0.274996 | 4.382836 | haematopoietic | blast phase chronic myeloid |
| | | | | | and lymphoid | leukaemia |
| | | | | | tissue | |
| MC116 | 0.004605 | 0.005215 | 0.252694 | 5.430331 | haematopoietic | B cell lymphoma |
| | | | | | and lymphoid | unspecified |
| | | | | | tissue | |
| COLO320HSR | 0.004609 | 0.00446 | 0.240303 | 0.900662 | large intestine | adenocarcinoma |
| LNCAP | 0.004648 | 0.003545 | 0.397386 | 21.5094 | prostate | adenocarcinoma |
| NOMO1 | 0.005012 | 0.003922 | 0.329287 | 8.171852 | haematopoietic | acute myeloid leukaemia |
| | | | | | and lymphoid | |
| | | | | | tissue | |
| KYSE150 | 0.005097 | 0.004407 | 0.305205 | 9.556776 | oesophagus | squamous cell carcinoma |
| KYSE270 | 0.005351 | 0.005115 | 0.306237 | 7.792632 | oesophagus | squamous cell carcinoma |
| C32 | 0.00536 | 0.005182 | 0.253167 | 1.007096 | skin | NS |
| SKMEL28 | 0.005562 | 0.005627 | 0.279597 | 4.919564 | skin | NS |
| WSUDLCL2 | 0.005671 | 0.004215 | 0.282886 | 0.634625 | haematopoietic | diffuse large B cell |
| | | | | | and lymphoid | lymphoma |
| | | | | | tissue | |
| MKN1 | 0.005748 | 0.004335 | 0.39452 | 17.75546 | stomach | mixed adenosquamous |
| | | | | | | carcinoma |
| NCIH520 | 0.005895 | 0.005059 | 0.323681 | 10.58477 | lung | squamous cell carcinoma |
| NCIH1836 | 0.006075 | 0.004881 | 0.333178 | 10.40721 | lung | small cell carcinoma |
| CHL1 | 0.006077 | 0.004679 | 0.349333 | 13.21706 | skin | NS |
| THP1 | 0.00615 | 0.004924 | 0.337139 | 12.47527 | haematopoietic | acute myeloid leukaemia |
| | | | | | and lymphoid | |
| | | | | | tissue | |
| AGS | 0.006186 | 0.005182 | 0.3536 | 14.1129 | stomach | adenocarcinoma |
| TOV112D | 0.006227 | 0.004264 | 0.3964 | 23.18819 | ovary | endometrioid carcinoma |
| CAOV3 | 0.006468 | 0.005254 | 0.390842 | 19.3273 | ovary | adenocarcinoma |
| KARPAS422 | 0.006525 | 0.005425 | 0.328997 | 8.084201 | haematopoietic | diffuse large B cell |
| | | | | | and lymphoid | lymphoma |
| | | | | | tissue | |
| NCIH358 | 0.006571 | 0.003053 | 0.433186 | 22.65882 | lung | bronchioloalveolar |
| | | | | | | adenocarcinoma |
| TE6 | 0.006677 | 0.004833 | 0.362521 | 14.02411 | oesophagus | squamous cell carcinoma |
| T.T | 0.006752 | 0.004366 | 0.432105 | 24.08447 | oesophagus | squamous cell carcinoma |
| NCIH524 | 0.006853 | 0.004977 | 0.361681 | 12.7766 | lung | small cell carcinoma |
| TE9 | 0.00689 | 0.003749 | 0.424717 | 25.30919 | oesophagus | squamous cell carcinoma |
| HSC4 | 0.006951 | 0.004332 | 0.43466 | 23.27648 | upper | squamous cell carcinoma |
| | | | | | aerodigestive tract | |
| MIAPACA2 | 0.007021 | 0.005283 | 0.332162 | 9.710695 | pancreas | ductal carcinoma |
| NCIH69 | 0.007089 | 0.00714 | 0.280211 | 1.270819 | lung | small cell carcinoma |
| KM12 | 0.007345 | 0.006303 | 0.348209 | 12.23928 | large intestine | adenocarcinoma |
| DV90 | 0.007364 | 0.005355 | 0.369777 | 11.78178 | lung | adenocarcinoma |
| FTC133 | 0.007491 | 0.004279 | 0.414551 | 22.3385 | thyroid | follicular carcinoma |
| NCIH1299 | 0.007498 | 0.005384 | 0.350839 | 12.64795 | lung | non-small cell carcinoma |
| NUGC3 | 0.00772 | 0.005627 | 0.38244 | 15.99752 | stomach | NS |
| NCIH1703 | 0.008047 | 0.004878 | 0.420393 | 21.7324 | lung | adenocarcinoma |
| KYSE410 | 0.008253 | 0.005469 | 0.415601 | 21.4639 | oesophagus | squamous cell carcinoma |
| NCIH2291 | 0.008405 | 0.007553 | 0.38223 | 13.48474 | lung | adenocarcinoma |
| TE5 | 0.008472 | 0.006638 | 0.387656 | 15.07094 | oesophagus | squamous cell carcinoma |
| U2OS | 0.009007 | 0.007712 | 0.406496 | 9.064236 | bone | NS |
| SW1463 | 0.009244 | 0.009177 | 0.340776 | 7.126189 | large intestine | adenocarcinoma |
| HL60 | 0.009317 | 0.008115 | 0.370342 | 9.446155 | haematopoietic | acute myeloid leukaemia |
| | | | | | and lymphoid | |
| | | | | | tissue | |
| NCIH716 | 0.009514 | 0.005771 | 0.379883 | 12.75198 | large intestine | adenocarcinoma |
| NCIH661 | 0.009732 | 0.009063 | 0.360829 | 9.074949 | lung | large cell carcinoma |
| SW480 | 0.009788 | 0.007726 | 0.409035 | 20.35763 | large intestine | adenocarcinoma |
| BFTC905 | 0.009914 | 0.008929 | 0.39887 | 13.73926 | urinary tract | transitional cell carcinoma |
| BT549 | 0.009983 | 0.005837 | 0.431039 | 20.54694 | breast | ductal carcinoma |
| NCIH508 | 0.010235 | 0.009159 | 0.337789 | 3.207058 | large intestine | adenocarcinoma |
| KU1919 | 0.010268 | 0.008773 | 0.395587 | 14.57044 | urinary tract | transitional cell carcinoma |
| COLO741 | 0.01036 | 0.006858 | 0.418613 | 18.78304 | skin | NS |
| MDAMB453 | 0.010402 | 0.008034 | 0.452361 | 21.95264 | breast | NS |
| LU65 | 0.010583 | 0.00719 | 0.409074 | 11.99716 | lung | non-small cell carcinoma |
| A375 | 0.010596 | 0.007623 | 0.440543 | 19.51092 | skin | NS |
| HCC70 | 0.01099 | 0.008561 | 0.413475 | 13.84525 | breast | ductal carcinoma |
| A101D | 0.011003 | 0.006914 | 0.468887 | 20.45562 | skin | NS |
| HLE | 0.011279 | 0.010386 | 0.383928 | 7.875661 | liver | hepatocellular carcinoma |
| RKO | 0.011534 | 0.008496 | 0.382689 | 11.05361 | large intestine | adenocarcinoma |
| NCIH460 | 0.011582 | 0.01108 | 0.371384 | 7.704677 | lung | large cell carcinoma |
| SNB19 | 0.011607 | 0.007011 | 0.467719 | 20.73493 | central nervous | astrocytoma Grade IV |
| | | | | | system | |
| HUH1 | 0.011768 | 0.010591 | 0.414451 | 11.68072 | liver | hepatocellular carcinoma |
| KMS11 | 0.012224 | 0.01094 | 0.391826 | 9.982711 | haematopoietic | plasma cell myeloma |
| | | | | | and lymphoid | |
| | | | | | tissue | |
| 647V | 0.01224 | 0.01115 | 0.391223 | 10.17987 | urinary tract | transitional cell carcinoma |
| TE4 | 0.012665 | 0.009106 | 0.394771 | 11.49063 | oesophagus | squamous cell carcinoma |
| NCIH1437 | 0.013132 | 0.009476 | 0.408233 | 14.97097 | lung | adenocarcinoma |
| HUH7 | 0.013955 | 0.012904 | 0.392615 | 7.054246 | liver | hepatocellular carcinoma |
| 143B | 0.014366 | 0.013944 | 0.358228 | 2.195792 | bone | NS |
| LS411N | 0.016662 | 0.016827 | 0.385144 | 6.414977 | large intestine | adenocarcinoma |
| SKMEL5 | 0.016833 | 0.014822 | 0.407428 | 8.227588 | skin | NS |
| KYSE30 | 0.002426 | 10 | 0.44339 | 38.58605 | oesophagus | squamous cell carcinoma |
| CAKI1 | 0.004429 | 10 | 0.531935 | 34.02825 | kidney | clear cell renal cell |
| | | | | | | carcinoma |
| HCC1599 | 0.005645 | 0.003823 | 0.463913 | 32.70157 | breast | ductal carcinoma |
| 5637 | 0.006107 | 0.002239 | 0.485659 | 33.00819 | urinary tract | NS |
| HCC1187 | 0.006507 | 0.003097 | 0.464968 | 33.99519 | breast | ductal carcinoma |
| COLO680N | 0.006972 | 0.004787 | 0.531063 | 33.76189 | oesophagus | squamous cell carcinoma |
| SKBR3 | 0.007137 | 0.004323 | 0.439603 | 26.58705 | breast | NS |
| NCIH1092 | 0.00716 | 0.003931 | 0.479044 | 26.40383 | lung | small cell carcinoma |
| OCUM1 | 0.00779 | 10 | 0.532422 | 32.42435 | stomach | diffuse adenocarcinoma |
| HSC2 | 0.007994 | 0.005088 | 0.451097 | 26.00789 | upper | squamous cell carcinoma |
| | | | | | aerodigestive tract | |
| NCIH1436 | 0.008754 | 10 | 0.495332 | 34.40202 | lung | small cell carcinoma |
| SKMEL1 | 0.009242 | 0.000536 | 0.442658 | 22.0664 | skin | NS |
| RD | 0.009294 | 0.004792 | 0.470348 | 27.41572 | soft tissue | embryonal |
| NCIH647 | 0.009841 | 0.006045 | 0.494182 | 28.28713 | lung | mixed adenosquamous |
| | | | | | | carcinoma |
| SCC4 | 0.010532 | 0.005568 | 0.461894 | 26.07786 | upper | squamous cell carcinoma |
| | | | | | aerodigestive tract | |
| NUGC4 | 0.010595 | 0.006625 | 0.464202 | 24.52784 | stomach | signet ring adenocarcinoma |
| HMCB | 0.011093 | 0.000815 | 0.504089 | 32.24977 | skin | NS |
| COLO205 | 0.011489 | 0.005969 | 0.483296 | 27.13083 | large intestine | adenocarcinoma |
| HEL | 0.011746 | 0.003598 | 0.526792 | 28.88964 | haematopoietic | acute myeloid leukaemia |
| | | | | | and lymphoid | |
| | | | | | tissue | |
| RPMI8226 | 0.01249 | 0.010401 | 0.436254 | 12.45893 | haematopoietic | plasma cell myeloma |
| | | | | | and lymphoid | |
| | | | | | tissue | |
| SW780 | 0.013009 | 0.011796 | 0.449063 | 13.9761 | urinary tract | transitional cell carcinoma |
| HCC1806 | 0.013019 | 0.005129 | 0.532503 | 36.86894 | breast | ductal carcinoma |
| NCIH2347 | 0.01306 | 10 | 0.577758 | 40.28995 | lung | adenocarcinoma |
| SHSY5Y | 0.013453 | 0.00432 | 0.492725 | 25.03904 | autonomic ganglia | NS |
| MKN45 | 0.013529 | 0.011004 | 0.442717 | 19.77129 | stomach | diffuse adenocarcinoma |
| HS578T | 0.013853 | 0.006902 | 0.537295 | 32.74703 | breast | ductal carcinoma |
| HT1080 | 0.014147 | 0.01275 | 0.543789 | 29.73724 | soft tissue | NS |
| HT55 | 0.014167 | 0.012668 | 0.470447 | 22.172 | large intestine | adenocarcinoma |
| MESSA | 0.014307 | 0.009003 | 0.493277 | 24.21537 | soft tissue | NS |
| PC3 | 0.014471 | 0.011814 | 0.502021 | 26.1466 | prostate | adenocarcinoma |
| U87MG | 0.014695 | 10 | 0.514049 | 38.45109 | central nervous | astrocytoma |
| | | | | | system | |
| CALU6 | 0.014859 | 0.011452 | 0.459539 | 18.31843 | lung | undifferentiated carcinoma |
| DAUDI | 0.014888 | 0.013395 | 0.538295 | 25.4109 | haematopoietic | Burkitt lymphoma |
| | | | | | and lymphoid | |
| | | | | | tissue | |
| KNS62 | 0.014901 | 0.012375 | 0.47611 | 18.83614 | lung | squamous cell carcinoma |
| SCABER | 0.015202 | 0.008931 | 0.568701 | 32.32311 | urinary tract | transitional cell carcinoma |
| CFPAC1 | 0.015268 | 0.011256 | 0.52266 | 29.21979 | pancreas | ductal carcinoma |
| NCIH1793 | 0.015288 | 0.008364 | 0.491588 | 27.47757 | lung | non-small cell carcinoma |
| TE10 | 0.016273 | 0.009297 | 0.529321 | 29.64652 | oesophagus | squamous cell carcinoma |
| NCIH1944 | 0.016307 | 10 | 0.580862 | 37.59181 | lung | non-small cell carcinoma |
| DETROIT562 | 0.016685 | 0.010403 | 0.587348 | 36.50766 | upper | NS |
| | | | | | aerodigestive tract | |
| NCIH522 | 0.01739 | 0.011534 | 0.444733 | 15.14236 | lung | non-small cell carcinoma |
| GRANTA519 | 0.017643 | 0.01487 | 0.468567 | 17.90564 | haematopoietic | mantle cell lymphoma |
| | | | | | and lymphoid | |
| | | | | | tissue | |
| DU145 | 0.01772 | 0.016588 | 0.432367 | 18.16451 | prostate | NS |
| SNU398 | 0.017992 | 0.009627 | 0.49108 | 25.8616 | liver | hepatocellular carcinoma |
| HLF | 0.018027 | 0.011534 | 0.465709 | 18.24934 | liver | hepatocellular carcinoma |
| NCIH1623 | 0.018862 | 0.00813 | 0.522025 | 30.06498 | lung | adenocarcinoma |
| SNU668 | 0.01888 | 0.012849 | 0.464013 | 16.0231 | stomach | signet ring adenocarcinoma |
| BXPC3 | 0.019388 | 0.008453 | 0.57381 | 36.5952 | pancreas | ductal carcinoma |
| NCIH1930 | 0.020006 | 0.006616 | 0.540233 | 36.32772 | lung | small cell carcinoma |
| HCT116 | 0.020431 | 0.004991 | 0.535649 | 35.91847 | large intestine | NS |
| NCIH1355 | 0.020573 | 10 | 0.559364 | 35.37581 | lung | adenocarcinoma |
| T84 | 0.020979 | 0.010096 | 0.492753 | 21.54238 | large intestine | adenocarcinoma |
| DMS273 | 0.022219 | 0.016264 | 0.481488 | 16.57774 | lung | small cell carcinoma |
| ECGI10 | 0.02244 | 10 | 0.568334 | 39.22919 | oesophagus | NS |
| 7860 | 0.022468 | 0.011656 | 0.504861 | 22.93315 | kidney | clear cell renal cell |
| | | | | | | carcinoma |
| HCC78 | 0.02392 | 0.012709 | 0.531509 | 23.40461 | lung | adenocarcinoma |
| NCIH2122 | 0.023938 | 0.016742 | 0.501905 | 18.69937 | lung | adenocarcinoma |
| OVISE | 0.023953 | 10 | 0.540484 | 41.60625 | ovary | clear cell carcinoma |
| LUDLU1 | 0.025828 | 0.012626 | 0.530668 | 28.03128 | lung | squamous cell carcinoma |
| GP2D | 0.026231 | 0.020627 | 0.506015 | 20.31771 | large intestine | adenocarcinoma |
| WM2664 | 0.027533 | 10 | 0.583762 | 37.49186 | skin | NS |
| PLCPRF5 | 0.02796 | 0.0164 | 0.510666 | 21.67484 | liver | hepatocellular carcinoma |
| SW620 | 0.02926 | 0.026311 | 0.465758 | 9.079409 | large intestine | adenocarcinoma |
| SKCO1 | 0.030585 | 10 | 0.595243 | 34.7288 | large intestine | adenocarcinoma |
| A549 | 0.032375 | 0.018492 | 0.573044 | 31.10718 | lung | non-small cell carcinoma |
| NCIH929 | 0.034475 | 0.020579 | 0.52854 | 24.32478 | haematopoietic | plasma cell myeloma |
| | | | | | and lymphoid | |
| | | | | | tissue | |
| ACHN | 0.034708 | 0.030334 | 0.453014 | 2.196602 | kidney | renal cell carcinoma |
| NCIH1975 | 0.034766 | 0.025525 | 0.528991 | 17.89159 | lung | non-small cell carcinoma |
| PANC0213 | 0.035795 | 0.021461 | 0.582932 | 32.945 | pancreas | NS |
| HT1376 | 0.036268 | 0.017852 | 0.551857 | 18.92436 | urinary tract | transitional cell carcinoma |
| HT29 | 0.037031 | 0.022837 | 0.574146 | 29.83252 | large intestine | adenocarcinoma |
| UMUC3 | 0.039504 | 0.021864 | 0.593428 | 35.04699 | urinary tract | transitional cell carcinoma |
| SNU761 | 0.039689 | 10 | 0.581426 | 38.06756 | liver | hepatocellular carcinoma |
| SKLU1 | 0.040003 | 0.008488 | 0.56624 | 38.28472 | lung | adenocarcinoma |
| HCC1954 | 0.040563 | 10 | 0.591175 | 43.62567 | breast | ductal carcinoma |
| NCIH23 | 0.042711 | 0.037736 | 0.622699 | 27.39399 | lung | non-small cell carcinoma |
| KATOIII | 0.043772 | 0.040387 | 0.490001 | 12.87902 | stomach | adenocarcinoma |
| A2058 | 0.046764 | 0.028087 | 0.56942 | 30.63873 | skin | NS |
| SKMES1 | 0.047461 | 0.024323 | 0.60692 | 30.65752 | lung | squamous cell carcinoma |
| SW48 | 0.050006 | 10 | 0.603764 | 37.9499 | large intestine | adenocarcinoma |
| HEPG2 | 0.052079 | 10 | 0.608647 | 37.75892 | liver | hepatocellular carcinoma |
| SNUC2A | 0.052512 | 0.024542 | 0.615038 | 34.41705 | large intestine | adenocarcinoma |
| LOVO | 0.057826 | 10 | 0.636191 | 47.95459 | large intestine | adenocarcinoma |
| HSC3 | 0.060326 | 10 | 0.6313 | 47.25644 | upper | squamous cell carcinoma |
| | | | | | aerodigestive tract | |
| JIMT1 | 0.061923 | 0.046579 | 0.551921 | 13.45549 | breast | ductal carcinoma |
| SW1710 | 0.063291 | 0.027254 | 0.5768 | 25.48455 | urinary tract | transitional cell carcinoma |
| RERFLCMS | 0.065898 | 0.023477 | 0.641498 | 38.15621 | lung | non-small cell carcinoma |
| HS294T | 0.066791 | 0.030717 | 0.600533 | 28.13572 | skin | NS |
| ASPC1 | 0.066825 | 0.018855 | 0.581226 | 32.80606 | pancreas | ductal carcinoma |
| PANC1 | 0.06858 | 0.051468 | 0.554273 | 12.02616 | pancreas | ductal carcinoma |
| CAPAN1 | 0.115609 | 10 | 0.639142 | 40.2242 | pancreas | ductal carcinoma |
| NCIH446 | 0.119463 | 0.014536 | 0.583736 | 24.17448 | lung | small cell carcinoma |
| HEP3B217 | 0.129298 | 10 | 0.644762 | 47.20301 | liver | hepatocellular carcinoma |
| NCIH2172 | 0.132833 | 10 | 0.68115 | 37.97151 | lung | non-small cell carcinoma |
| SKMEL2 | 0.13968 | 0.026999 | 0.639177 | 35.37217 | skin | NS |
| SNU5 | 0.146396 | 0.082367 | 0.683652 | 32.15273 | stomach | undifferentiated |
| | | | | | | adenocarcinoma |
| NCIH2228 | 0.178529 | 10 | 0.686403 | 44.26922 | lung | adenocarcinoma |
| BICR18 | 0.19427 | 0.186872 | 0.618995 | 4.76155 | upper | squamous cell carcinoma |
| | | | | | aerodigestive tract | |
| GI1 | 0.28744 | 0.295062 | 0.623711 | 9.190768 | central nervous | gliosarcoma |
| | | | | | system | |
| MCF7 | 0.434714 | 10 | 0.659214 | 43.69748 | breast | NS |
| SKMEL3 | 0.472006 | 10 | 0.620722 | 46.89633 | skin | NS |
| WM115 | 0.477738 | 0.002502 | 0.609719 | 36.92872 | skin | NS |
| COLO679 | 2.044655 | 2.344991 | 0.502041 | 24.71872 | skin | NS |
| SNGM | 4.901703 | 4.138108 | 0.916457 | 26.87739 | endometrium | adenocarcinoma |
| NCIH2171 | 10 | 10 | 0.7416 | 16.52996 | lung | small cell carcinoma |
| SUPT1 | 3.3333 | 10 | 0.691296 | 48.11215 | haematopoietic | acute lymphoblastic T cell |
| | | | | | and lymphoid | leukaemia |
| | | | | | tissue | |
| KNS81 | 10 | 10 | 0.630939 | 58.5801 | central nervous | astrocytoma Grade IV |
| | | | | | system | |
| MPP89 | 10 | 10 | 0.638426 | 51.42635 | pleura | NS |
| SNU886 | 10 | 10 | 0.642533 | 48.34561 | liver | hepatocellular carcinoma |
| COLO829 | 10 | 10 | 0.655706 | 47.96395 | skin | NS |
| KALS1 | 10 | 10 | 0.65593 | 49.78685 | central nervous | NS |
| | | | | | system | |
| SNU878 | 10 | 10 | 0.664801 | 52.14968 | liver | hepatocellular carcinoma |
| GAK | 10 | 10 | 0.664855 | 47.69716 | NOT IN CCLE | NOT IN CCLE |
| OSRC2 | 10 | 10 | 0.665792 | 52.3524 | kidney | renal cell carcinoma |
| RCM1 | 10 | 10 | 0.671054 | 42.54585 | large intestine | adenocarcinoma |
| NCIH1734 | 10 | 10 | 0.673415 | 54.90475 | lung | adenocarcinoma |
| DMS153 | 10 | 10 | 0.673713 | 50.81473 | lung | small cell carcinoma |
| J82 | 10 | 10 | 0.674852 | 48.70006 | urinary tract | transitional cell carcinoma |
| BICR22 | 10 | 10 | 0.682919 | 54.75925 | upper | squamous cell carcinoma |
| | | | | | aerodigestive tract | |
| SW1990 | 10 | 10 | 0.689389 | 49.22702 | pancreas | ductal carcinoma |
| HCC1143 | 10 | 10 | 0.69059 | 50.38194 | breast | ductal carcinoma |
| HCC2935 | 10 | 10 | 0.692215 | 50.08429 | lung | non-small cell carcinoma |
| KYSE70 | 10 | 10 | 0.694176 | 50.18949 | oesophagus | squamous cell carcinoma |
| MDAMB468 | 10 | 10 | 0.697183 | 50.86583 | breast | NS |
| NMCG1 | 10 | 10 | 0.701293 | 47.05987 | central nervous | NS |
| | | | | | system | |
| SKHEP1 | 10 | 10 | 0.70906 | 44.3029 | liver | adenocarcinoma |
| SW837 | 10 | 10 | 0.710855 | 46.73384 | large intestine | adenocarcinoma |
| A204 | 10 | 10 | 0.713626 | 58.90593 | soft tissue | NS |
| HCC1419 | 10 | 10 | 0.720097 | 52.41521 | breast | ductal carcinoma |
| BT474 | 10 | 10 | 0.720573 | 53.87865 | breast | ductal carcinoma |
| MEWO | 10 | 10 | 0.723674 | 56.56317 | skin | NS |
| MDAMB231 | 10 | 10 | 0.726163 | 55.94067 | breast | NS |
| CALU3 | 10 | 10 | 0.727683 | 50.88262 | lung | adenocarcinoma |
| SKNSH | 10 | 10 | 0.729486 | 53.89154 | autonomic ganglia | NS |
| ONS76 | 10 | 10 | 0.73362 | 54.63371 | central nervous | NS |
| | | | | | system | |
| MDAMB175VII | 10 | 10 | 0.738455 | 60.66071 | breast | ductal carcinoma |
| SKMEL31 | 10 | 10 | 0.739322 | 59.48678 | skin | NS |
| DMS53 | 10 | 10 | 0.742606 | 49.07034 | lung | small cell carcinoma |
| SNUC1 | 10 | 10 | 0.747771 | 55.45382 | large intestine | adenocarcinoma |
| NCIH1573 | 10 | 10 | 0.748221 | 53.57475 | lung | adenocarcinoma |
| NCIH1435 | 10 | 10 | 0.761242 | 60.04075 | lung | non-small cell carcinoma |
| NIHOVCAR3 | 10 | 10 | 0.777131 | 59.47137 | ovary | NS |
| SCC25 | 10 | 10 | 0.778864 | 62.3079 | upper | squamous cell carcinoma |
| | | | | | aerodigestive tract | |
| NCIH441 | 10 | 10 | 0.78042 | 67.48763 | lung | adenocarcinoma |
| HCC1428 | 10 | 10 | 0.781839 | 59.9889 | breast | NS |
| VCAP | 10 | 10 | 0.782184 | 62.59825 | prostate | adenocarcinoma |
| NCIH1838 | 10 | 10 | 0.785788 | 60.0585 | lung | non-small cell carcinoma |
| SKMEL24 | 10 | 10 | 0.7882 | 58.11712 | skin | NS |
| LU99 | 10 | 10 | 0.78999 | 64.65551 | lung | large cell carcinoma |
| NCIH2009 | 10 | 8.019371 | 0.791426 | 55.69455 | lung | adenocarcinoma |
| RPMI7951 | 10 | 10 | 0.806669 | 66.30924 | skin | NS |
| NCIH596 | 10 | 10 | 0.815117 | 61.33612 | lung | mixed adenosquamous |
| | | | | | | carcinoma |
| TE8 | 10 | 10 | 0.816021 | 72.1307 | oesophagus | squamous cell carcinoma |
| SW1271 | 10 | 10 | 0.817932 | 65.37809 | lung | small cell carcinoma |
| C2BBE1 | 10 | 10 | 0.830329 | 59.34028 | large intestine | adenocarcinoma |
| CAL27 | 10 | 10 | 0.833497 | 74.67924 | upper | squamous cell carcinoma |
| | | | | | aerodigestive tract | |
| MDAMB157 | 10 | 10 | 0.836176 | 64.71391 | breast | ductal carcinoma |
| UACC812 | 10 | 6.947929 | 0.841822 | 62.67078 | breast | ductal carcinoma |
| MDAMB436 | 10 | 10 | 0.843786 | 70.42433 | breast | NS |
| NCIH1395 | 10 | 10 | 0.854187 | 70.68192 | lung | adenocarcinoma |
| HS739T | 10 | 10 | 0.868583 | 78.34986 | breast | NS |
| KURAMOCHI | 10 | 10 | 0.874218 | 73.84661 | ovary | undifferentiated carcinoma |
| NCIH1650 | 10 | 10 | 0.874566 | 68.07063 | lung | bronchioloalveolar |
| | | | | | | adenocarcinoma |
| CCFSTTG1 | 10 | 10 | 0.879034 | 78.89955 | central nervous | astrocytoma |
| | | | | | system | |
| CAPAN2 | 10 | 10 | 0.880831 | 75.01844 | pancreas | ductal carcinoma |
| HCC827 | 10 | 10 | 0.881358 | 74.84 | lung | adenocarcinoma |
| KASUMI1 | 10 | 10 | 0.882289 | 81.08059 | haematopoietic | acute myeloid leukaemia |
| | | | | | and lymphoid | |
| | | | | | tissue | |
| HS852T | 10 | 10 | 0.883001 | 78.51343 | skin | NS |
| HT144 | 10 | 10 | 0.89125 | 81.60806 | skin | NS |
| HCC1937 | 10 | 10 | 0.9094 | 80.9493 | breast | ductal carcinoma |
| BT20 | 10 | 10 | 0.911675 | 78.79204 | breast | ductal carcinoma |
| SW579 | 10 | 10 | 0.920274 | 79.98259 | thyroid | anaplastic carcinoma |
| HS695T | 10 | 10 | 0.923787 | 74.11092 | skin | NS |
| T24 | 10 | 10 | 0.928907 | 75.74279 | urinary tract | transitional cell carcinoma |
| TE1 | 10 | 10 | 0.937963 | 84.54246 | oesophagus | squamous cell carcinoma |
| MDAMB415 | 10 | 10 | 0.941323 | 83.39028 | breast | NS |
| SCC9 | 10 | 10 | 0.942441 | 69.24641 | upper | squamous cell carcinoma |
| | | | | | aerodigestive tract | |
| SCC15 | 10 | 10 | 0.948945 | 90.39119 | upper | squamous cell carcinoma |
| | | | | | aerodigestive tract | |
| KG1 | 10 | 10 | 0.959161 | 80.43661 | haematopoietic | acute myeloid leukaemia |
| | | | | | and lymphoid | |
| | | | | | tissue | |
| SW1417 | 10 | 10 | 0.963372 | 91.96064 | large intestine | adenocarcinoma |
| NCIH1563 | 10 | 10 | 0.965176 | 93.507 | lung | adenocarcinoma |
| DAOY | 10 | 10 | 0.968421 | 83.40166 | central nervous | NS |
| | | | | | system | |
| HUH28 | 10 | 10 | 0.98625 | 92.15381 | biliary tract | NS |
| HCC38 | 10 | 10 | 0.98639 | 93.61212 | breast | ductal carcinoma |
| MALME3M | 10 | 10 | 0.986698 | 94.08447 | skin | NS |
| SKOV3 | 10 | 10 | 0.990095 | 96.03318 | ovary | adenocarcinoma |
| G361 | 10 | 10 | 1.043507 | 89.52446 | skin | NS |
| SAOS2 | 10 | 10 | 1.080519 | 92.52133 | bone | NS |
| TE15 | 10 | 10 | 1.084416 | 89.04183 | oesophagus | squamous cell carcinoma |
| HS839T | 10 | 10 | 1.09737 | 101.2434 | skin | NS |
| HS688AT | 10 | 10 | 1.105351 | 98.46712 | skin | NS |
| MDAMB361 | 10 | 10 | 1.116565 | 94.55241 | breast | NS |
| CALU1 | 10 | 10 | 1.217208 | 118.4694 | lung | squamous cell carcinoma |
| NCIH1755 | 10 | 10 | 1.289838 | 123.3293 | lung | adenocarcinoma |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to milling.

2. The pharmaceutical composition of claim 1, wherein the milling is performed with a ball mill.

3. The pharmaceutical composition of claim 1, wherein the milling is performed with a roller mill or a high energy mill.

4. The pharmaceutical composition of claim 1, 2, or 3, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt exhibits a particle size less than 1000 nanometers.

5. The pharmaceutical composition of any one of claims 1-4, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt exhibits a particle size from about 50 nanometers to about 1000 nanometers.

6. The pharmaceutical composition of any one of claims 1-5, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt exhibits a particle size from about 50 nanometers to about 100 nanometers, from about 100 nanometers to about 200 nanometers, from about 200 nanometers to about 300 nanometers, from about 300 nanometers to about 400 nanometers, from about 400 nanometers to about 500 nanometers, from about 500 nanometers to about 600 nanometers, from about 700 nanometers to about 800 nanometers, from about 800 nanometers to about 900 nanometers, or from about 900 nanometers to about 1000 nanometers.

7. The pharmaceutical composition of any one of claims 1-5, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt exhibits a particle size from about 150 nanometers to about 300 nanometers.

8. The pharmaceutical composition of any one of claims 1-7, wherein the particle size does not increase upon storage.

9. The pharmaceutical composition of any one of claims 1-7, wherein the particle size does not increase more than 5% upon storage.

10. The pharmaceutical composition of any one of claims 1-7, wherein the particle size does not increase more than 10% upon storage.

11. The pharmaceutical composition of any one of claims 1-7, wherein the particle size does not increase more than 15% upon storage.

12. The pharmaceutical composition of any one of claims 1-11, wherein the at least one pharmaceutically acceptable excipient is a solubilizing agent.

13. The pharmaceutical composition of claim 12, wherein the solubilizing agent is a polyethylene glycol (PEG).

14. The pharmaceutical composition of claim 13, wherein the PEG is selected from PEG 200, PEG 300, PEG 400, PEG 500, or PEG 600.

15. The pharmaceutical composition of any one of claims 1-14, wherein the composition further comprises a stabilizer.

16. The pharmaceutical composition of claim 15, wherein the stabilizer is selected from the group consisting of hydroxy propyl methyl cellulose (HPMC), hydroxypropyl cellulose (HPC), polyvinylpyrrolidone (PVP) or poloxamer.

17. The pharmaceutical composition of any one of claims 1-16, wherein the composition comprises:
(a) 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt from about 0.1% (w/w) to about 0.2% (w/w);
(b) hydroxypropyl cellulose from about 0.91% (w/w) to about 1.5% (w/w);
(c) PEG 400 from about 5.0% (w/w) to about 10.0% (w/w); and
(d) water from about 89.3% (w/w) to about 94.4% (w/w).

18. The pharmaceutical composition of claim 17, wherein the composition comprises:
(a) 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt 0.17% (w/w);
(b) hydroxypropyl cellulose 0.91% (w/w);
(c) PEG 400 8.32% (w/w); and
(d) water 90.49% (w/w).

19. The pharmaceutical composition of claim 17 or 18, wherein the composition is a tablet dosage form or a capsule dosage form.

20. A pharmaceutical composition comprising 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt and at least one pharmaceutically acceptable excipient, wherein the 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt has been subjected to lyophilization, or spray drying, or a combination thereof.

21. The pharmaceutical composition of claim 20, wherein the at least one pharmaceutically acceptable excipient is a solubilizing agent.

22. The pharmaceutical composition of claim 21, wherein the solubilizing agent is a polyethylene glycol (PEG) selected from PEG 200, PEG 300, PEG 400, PEG 500, or PEG 600.

23. The pharmaceutical composition of claim 22, wherein the PEG is selected from PEG 1000, PEG 1500, or PEG 2000.

24. The pharmaceutical composition of any one of claims 20-23, wherein the at least one pharmaceutically acceptable excipient is a stabilizer.

25. The pharmaceutical composition of claim 24, wherein the stabilizer is selected from copovidone, or kollidon VA64.

26. The pharmaceutical composition of any one of claims 20-25, wherein the at least one pharmaceutically acceptable excipient is a disintegrant.

27. The pharmaceutical composition of claim 26, wherein the disintegrant is selected from crospovidone, or kollidon CL.

28. The pharmaceutical composition of any one of claims 20-27, wherein the composition comprises:
(a) 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt from about 10% (w/w) to about 20% (w/w);
(b) Kollidon VA 64 from about 70% (w/w) to about 80% (w/w);
(c) PEG 1500 from about 2% (w/w) to about 7% (w/w); and
(d) Kollidon CL from about 5% (w/w) to about 15% (w/w).

29. The pharmaceutical composition of claim 28, wherein the composition comprises:
(a) 3-({[(4R)-7-{methyl[4-(propan-2-yl)phenyl]amino}-3,4-dihydro-2H-1-benzopyran-4-yl]methyl}amino)pyridine-4-carboxylic acid, L-lysine salt 15% (w/w);
(b) Kollidon VA64 77% (w/w);
(c) PEG 1500 5% (w/w); and
(d) Kollidon CL 10% (w/w).

30. The pharmaceutical composition of any one of claims 20-29, wherein the composition is a tablet dosage form or a capsule dosage form.

31. The pharmaceutical composition of any one of claims 20-30, wherein the composition exhibits long term stability.

32. A method of treating a cancer in a patient in need thereof, comprising administering to the patient a pharmaceutical composition of any one of claims 1-31.

33. A method of treating a cancer in a patient in need thereof, comprising administering to the patient a therapeutically effective amount of a pharmaceutical composition of any one of claims 1-32.

34. The method of claim 32 or 33, wherein the cancer is selected from a hematologic or a solid malignancy.

35. The method of claim 32 or 33, wherein the cancer is selected from the group consisting of colorectal cancer, esophageal cancer, triple negative breast cancer, gastric cancer, lymphoma, gastric adenocarcinoma, diffuse large B-cell non-Hodgkin's lymphoma, acute T-cell leukemia, esophageal squamous cell carcinoma, multiple myeloma, acute myeloid leukemia, colorectal adenocarcinoma, colorectal carcinoma, pancreatic cancer, pancreatic carcinoma, breast carcinoma, and T-cell acute lymphoblastic leukemia.

36. The method of claim 32 or 33, wherein the cancer patient has been diagnosed with colorectal cancer.

37. The method of claim 32 or 33, wherein the cancer patient has been diagnosed with esophageal cancer.

38. The method of claim 32 or 33, wherein the cancer patient has been diagnosed with triple negative breast cancer.

39. The method of claim 32 or 33, wherein the cancer patient has been diagnosed with gastric cancer.

40. The method of claim 32 or 33, wherein the cancer patient has been diagnosed with lymphoma.

41. The method of claim 32 or 33, wherein the cancer patient has been diagnosed with gastric adenocarcinoma.

42. The method of claim 32 or 33, wherein the cancer patient has been diagnosed with diffuse large B-cell non-Hodgkin's lymphoma.

43. The method of claim 32 or 33, wherein the cancer patient has been diagnosed with acute T-cell leukemia.

44. The method of claim 32 or 33, wherein the cancer patient has been diagnosed with esophageal squamous cell carcinoma.

45. The method of claim 32 or 33, wherein the cancer patient has been diagnosed with multiple myeloma.

46. The method of claim 32 or 33, wherein the cancer patient has been diagnosed with acute myeloid leukemia.

47. The method of claim 32 or 33, wherein the cancer patient has been diagnosed with colorectal adenocarcinoma.

48. The method of claim 32 or 33, wherein the cancer patient has been diagnosed with colorectal carcinoma.

49. The method of claim 32 or 33, wherein the cancer patient has been diagnosed with pancreatic cancer.

50. The method of claim 32 or 33, wherein the cancer patient has been diagnosed with pancreatic carcinoma.

51. The method of claim 32 or 33, wherein the cancer patient has been diagnosed with breast carcinoma.

52. The method of claim 32 or 33, wherein the cancer patient has been diagnosed with T-cell acute lymphoblastic leukemia.

53. The method of claim 32 or 33, wherein the cancer patient has been diagnosed with a cancer selected from lung cancer, small cell lung cancer, non-small cell lung cancer, large cell lung cancer, lung adenocarcinoma, lung squamous cell carcinoma, lung small cell carcinoma, lung large cell carcinoma, or bronchioloalveolar adenocarcinoma.

54. The method of claim 32 or 33, wherein the cancer patient has been diagnosed with a cancer selected from acute lymphoblastic B-cell leukemia, mantle cell lymphoma, plasma cell myeloma, diffuse large B-cell lymphoma, B-cell lymphoma, Burkitt lymphoma, blast phase chronic myeloid leukemia.

55. The method of claim 32 or 33, wherein the cancer patient has been diagnosed with a cancer selected from intestinal cancer, intestinal adenocarcinoma, squamous cell carcinoma of the upper digestive tract.

56. The method of claim 32 or 33, wherein the cancer patient has been diagnosed with a cancer selected from stomach cancer, stomach signet ring adenocarcinoma, adenocarcinoma of the stomach, or adenosquamous carcinoma.

57. The method of claim 32 or 33, wherein the cancer patient has been diagnosed with a cancer selected from ovarian cancer, ovarian endometrioid carcinoma, ovarian clear cell carcinoma, ovarian adenocarcinoma, endometrial cancer, endometrial adenocarcinoma, prostate cancer, or prostate adenocarcinoma.

58. The method of claim 32 or 33, wherein the cancer patient has been diagnosed with skin cancer.

59. The method of claim 32 or 33, wherein the cancer patient has been diagnosed with a cancer selected from thyroid cancer, or thyroid follicular carcinoma.

60. The method of claim 32 or 33, wherein the cancer patient has been diagnosed with a cancer selected from breast cancer, or breast ductal carcinoma.

61. The method of claim 32 or 33, wherein the cancer patient has been diagnosed with a cancer selected from liver cancer, or hepatocellular carcinoma.

62. The method of claim 32 or 33, wherein the cancer patient has been diagnosed with a cancer selected from a CNS cancer, astrocytoma grade IV, or gliosarcoma.

63. The method of claim 32 or 33, wherein the cancer patient has been diagnosed with bone cancer.

64. The method of claim 32 or 33, wherein the cancer patient has been diagnosed with a cancer selected from kidney cancer, clear cell renal cell carcinoma, renal cell carcinoma, urinary tract cancer, or urinary tract transitional cell carcinoma.

65. The method of any one of claims 34-64, wherein the cancer is relapsed after prior therapy, refractory to prior therapy, or acquired resistance to prior therapy.
